(19) United States
(12) Patent Application Publication
(10) Pub. No.: US 2012/0074014 A1
(43) Pub. Date: Mar. 29, 2012

(54) PRODUCT TYPICALLY BASED ON SALT OF PEROXYMONOSULFURIC ACID AND SUITABLE FOR MEDICINAL USAGE, AND ASSOCIATED PRODUCT FABRICATION

(75) Inventors: David Van Tran, San Jose, CA (US); David Nguyen Tran, San Jose, CA (US)

(73) Assignee: LuTran Industries, Inc.

(21) Appl. No.: 13/047,742

(22) Filed: Mar. 14, 2011

Related U.S. Application Data
(60) Provisional application No. 61/386,928, filed on Sep. 27, 2010.

(51) Int. Cl. A61B 19/02; B23P 19/00; A61K 33/42; A61K 9/14; C07C 409/44; A61K 31/327

(52) U.S. Cl. 206/438; 562/1; 514/578; 424/605; 424/400; 29/428

(57) ABSTRACT

Products based on salt of peroxymonosulfuric acid are suitable for treating or/and preventing diseases and other debilitating medical conditions caused by bacterial, eukaryotic, prion, and viral pathogens and by non-pathogenic inflammation. The products may alternatively be based on inorganic halide and an oxidizing agent reactable in water with the inorganic halide to generate hypohalite ions. In addition, the products can be employed in other applications such as commercial and industrial applications.

### CROSS-REFERENCE TO RELATED APPLICATION

This claims priority to U.S. provisional patent application 61/386,928, filed 27 Sep. 2010, the contents of which are incorporated by reference to the extent not repeated herein. This is also related to U.S. patent application Ser. No. 12/726,326, filed 17 Mar. 2009, the contents of which are likewise incorporated by reference to the extent not repeated herein.

### FIELD OF USE

This relates to products suitable for treating and preventing debilitating conditions, including debilitating medical conditions of humans. This also relates to manufacturing such products.

### BACKGROUND OF THE INVENTION

Inflammation is caused by tissue injury consisting of complex reactions involving vascular and connective tissues. Tissue damage may result from microbial invasion, auto-immune processes, tissue infection, allograft rejection, and such hurtful and/or destructive external influences as heat, cold, radiant energy, electrical or chemical stimuli, and mechanical trauma. Tissue damage may involve any part of the human body such as the joints (arthritis), bowels (inflammatory bowel disease), and lungs (pulmonary inflammation). Whatever the cause or bodily site, inflammatory responses to tissue damage are quite similar, consisting of complicated functional and cellular adjustments involving microcirculation, fluid shifts, and inflammatory cells (leukocytes). When tissue damage occurs, soluble chemical substances are elaborated which initiate the inflammatory response. Inflammation can be mild and self-limited or prolonged and seriously debilitating and chronic.

Numerous drugs have been developed to fight inflammation in humans. The most prominent in current treatment are anti-inflammatory steroidal drugs, corticosteroids and non-steroidal anti-inflammatory drugs such as salicylates. While these drugs are generally effective, they often have adverse side effects.

A pathogen is an infectious biological agent, sometimes referred to as a germ, which causes disease or illness to its host. Many medical advances, such as vaccination, antibiotics, and fungicides, have been used to safeguard against infection by pathogens. Nevertheless, pathogens continue to threaten human life. Primary pathogens are bacteria, eukaryotes, prions, and viruses.

Bacteria constitute one of the smallest organisms containing all the material required for growth and self-replication. Bacterial infections can be treated with antibiotics, classified as bacteriocidal if they kill bacteria and as bacteriostatic if they prevent the bacteria from multiplying so the human immune system can overcome them. There are many types of antibiotics. Each type of antibiotic inhibits a process whose pathogen is different from that found in the host. The effectiveness of individual antibiotics varies with the location of the infection, the ability of the antibiotic to reach the site of infection, and the ability of the bacteria to resist or inactivate the antibiotic. The adverse side effects of antibiotics are varied, and range from fever and nausea to major allergic reactions.

Fungi are eukaryotic pathogens similar to bacteria. Spores are metabolic byproducts of the life cycle of some bacteria and fungi. Bacteria produce endospores located within the cytoplasm of the parental cells. Fungi produce a variety of exospores. Spores are highly resistant to physical and chemical agents.

In medical parasitology, the term “parasite” means a eukaryotic pathogenic organism. Hence, protozoan and metazoan infectious agents are classified as parasites whereas bacteria and viruses are not. Many parasites, such as protozoa, fleas, and worms (helminths), carry disease or cause sores or lesions which can become infected.

Parasites live on or in the host from which it gets some or all of its nourishment. Parasites are generally harmful to their hosts. The damage ranges widely from minor inconvenience to debilitating or fatal disease. An ectoparasite, such as a louse, tick, or leech, lives or feeds on the outer surface of the host's body. Ectoparasites do not usually cause disease themselves. However, they are frequently a vector of disease. For example, tick parasites transmit organisms that can cause disease. An endoparasite lives inside the body of its host. Endoparasites include organisms such as tapeworms, hookworms, and trypanosomes that live within the host's organs or tissues as well as organisms such as sporozoans that invade the host's cells.

A prion is an infectious agent generally made solely of protein and lacking nucleic acid. Prions are believed to infect and propagate by refolding abnormally into a structure which converts normal molecules of the protein into an abnormally structured form. Prions are generally quite resistant to denaturation by protease, heat, radiation, and formalin treatments, although potency or infectivity may be reduced.

A virus consists of a single nucleic acid, either deoxyribonucleic acid (“DNA”) or ribonucleic acid (“RNA”), and a protein shell or coat surrounding the nucleic acid. A complete viral particle is called a virion. A virus uses the machinery of a host cell to reproduce and resides within the host cell. Consequently, viruses are difficult to eliminate without killing the host cells. It is believed that viral infections trigger inflammatory responses which do not respond to anti-viral drugs. Patients often ask for, and physicians often prescribe, antibiotics. While antibiotics destroy or prevent the growth of bacteria, antibiotics are useless in treating viral (and fungal) infections. Their misuse in treating viral diseases is one of the causes of antibiotic resistance to bacteria.

Sporkenbach et al. (“Sporkenbach”), U.S. Pat. No. 4,404,191, discloses a viricide technique for inactivating viruses on animate and inanimate surfaces by contacting the surfaces with a salt of peroxymonosulfuric acid (H₂SO₅) commonly known as Caro's acid. The peroxymonosulfuric acid salt, applied from an aqueous solution, can be a salt of an alkali metal such as potassium, sodium, or lithium, a salt of an alkaline earth metal such as calcium or magnesium, or an ammonium salt. Sporkenbach preferably employs KHSO₅ as the peroxymonosulfuric acid salt. KHSO₅ is provided from the mixed triple salt having the chemical formula 2KHSO₅.KHSO₄.K₂SO₄ where KHSO₄ is potassium hydrogen sulfate and K₂SO₄ is potassium sulfate sometimes referred to as dipotassium sulfate.

KHSO₅ and 2KHSO₅.KHSO₄.K₂SO₄ each have multiple chemical names. Both KHSO₅ and 2KHSO₅.KHSO₄.K₂SO₄ are commonly referred to as “potassium monopersulfate”. To avoid confusion, KHSO₅ is referred to herein as “potassium hydrogen peroxymonosulfate” or simply “potassium peroxymonosulfate”. 2KHSO₅.KHSO₄.K₂SO₄ is referred to herein as “potassium monopersulfate triple salt” or sometimes simply as “potassium monopersulfate”.

Sporkenbach identifies poliovirus, coxsackie virus, simian vacuolating virus 40 and adenovirus as being inactivated by potassium hydrogen peroxymonosulfate. Poliovirus causes poliomyelitis. There are two forms of coxsackie virus, type A and type B. Coxsackie A virus causes hand, foot, and mouth disease, acute haemorrhagic conjunctivitis, herpangina, and aseptic meningitis which includes viral meningitis. Coxsackie B virus causes pleurodynia (Bornholm disease) and can induce aseptic meningitis and diabetes mellitus type 1. Simian vacuolating virus 40 can cause tumors and cancer. Adenovirus generally produces infections in the upper respiratory tract. Adenovirus infections often appear as gastroenteritis, conjunctivitis, cystitis, and rash illness. Symptoms of respiratory illness caused by adenovirus infection include acute viral nasopharyngitis, pneumonia, croup, and bronchitis.

Sporkenbach discloses that its viricide technique can disinfect inanimate surfaces such as walls, floors and work surfaces, hospital utensils, and surgical and dental instruments in industrial, domestic, and medical environments and animate surfaces such as the skin of human and non-human animals during presurgical preparation in human and veterinary medicine. While Sporkenbach's viricide technique may prevent the diseases caused by the preceding viruses from being contracted, Sporkenbach's technique does not help already-infected people recover from those diseases.

Auchincloss, U.S. Pat. No. 4,822,512, discloses a dry water-soluble biocide for inactivating certain types of viruses, bacteria, and mold on non-human animals, specifically chickens, pigs, cows, and horses. The biocide contains (a) 0.01 to 5 parts by weight of a water-soluble inorganic halide, (b) 25 to 60 parts by weight of an oxidizing agent which reacts, in aqueous solution, with the halide to generate hypohalite ions, (c) 3 to 8 parts by weight of sulfamic acid, and (d) 10 to 30 parts by weight of an anhydrous alkali metal phosphate subject to the total parts totaling 100. The biocide may include up to 20 parts by weight of a non-reducing organic acid and up to 20 parts by weight of an organic surfactant.

The preferred oxidizing agent in Auchincloss's biocide is a persulfate or a peroxyphthalate. A persulfate is a sulfur-oxygen-containing compound having more oxygen than a normal sulfate. The additional oxygen in a persulfate is present in the form of one or more peroxide units, a peroxide being a chemical compound which includes an oxygen-oxygen single bond. The main types of persulfates are peroxymonosulfates and peroxydisulfates. A peroxyphthalate is a compound having more oxygen than a normal phthalate, the additional oxygen likewise being present in the form of one or more peroxide units.

Auchincloss's oxidizing agent is normally potassium monopersulfate triple salt, i.e., 2KHSO₅.KHSO₄.K₂SO₄. The halide is preferably sodium chloride but can be potassium chloride, potassium bromide, potassium iodide, sodium bromide, or sodium iodide. The organic acid is preferably malic or succinic acid. The alkali metal phosphate can be any one of sodium hexametaphosphate, monosodium phosphate, disodium phosphate, trisodium phosphate, tetrasodium pyrophosphate, monopotassium phosphate, dipotassium phosphate, tripotassium phosphate, and tetrapotassium pyrophosphate. According to Auchincloss, one embodiment of the biocide apparently consisted of 1.5 parts of sodium chloride, 50 parts of potassium monopersulfate triple salt, 10 parts of sulfamic acid, 5 parts of malic or succinic acid, 18.5 parts of sodium hexametaphosphate and (possibly) other alkali metal phosphate, and 15 parts of sodium dodecylbenzene sulfonate as the surfactant.

Auchincloss reported generally good results in variously using its biocide to disinfect chickens, pigs, cows, and horses. Auchincloss also reported that chickens can drink the biocide (apparently without harm), the biocide is not a skin or eye irritant, it is possible to bathe in the biocide (likewise apparently without harm), and the biocide can be sprayed in occupied rooms without causing discomfort.

Potassium peroxymonosulfate triple salt used by Sporkenbach and Auchincloss is commercially available from various sources including E.I. Dupont de Nemours and Company under the trade name Oxone and United Initiators under the trade name Caroat. Potassium hydrogen peroxymonosulfate, the principal component of potassium peroxymonosulfate triple salt, is a strong oxidizing agent. For instance, potassium hydrogen peroxymonosulfate can convert halide ions into halogens, ferrous ions into ferric ions, manganous ions into manganic ions, and hydrogen peroxide into oxygen. Potassium hydrogen peroxymonosulfate can also initiate the free radical polymerization of vinyl monomers such as vinyl acetate, ethyl acrylate, and acrylonitrile. In addition to the uses mentioned above, potassium hydrogen peroxymonosulfate serves as a bleaching agent in denture cleansers, toilet-bowl cleaners, and laundry/dry bleaches.

Valliéres, U.S. Pat. No. 5,186,946, discloses a disinfectant reportedly effective against bacteria, fungi, bacterial and fungal spores, and viruses. Somewhat similar to Auchincloss's composition, Valliéres' disinfectant consists of 60 to 90 weight % potassium hydrogen peroxymonosulfate, 2-10 weight % malic acid, 2-6 weight % sulfamic acid, 0.25-3 weight % ethylene diamine tetraacetic acid disodium salt, and 1-15 weight % alkylated ether of polyethylene glycol surfactant. Different from Auchincloss, Valliéres avoids the chlorine present in the sodium chloride preferably used by Auchincloss to implement the inorganic halide in Auchincloss's biocide. Valliéres states that its disinfectant is to be used for cleaning instruments, floors, and bedding in hospitals, bio-medical research centers, health centers, veterinary hospitals, and clinics.

Potassium hydrogen peroxymonosulfate is also used for removing chloramines in swimming pools. Regarding swimming pools, Lightcap et al. (“Lightcap”), U.S. Pat. No. 7,560,033 B2, discloses an anhydrous composition formed with potassium hydrogen peroxymonosulfate and an active halogen agent for sanitizing water in recirculating water systems such as swimming pools. The active halogen agent consists of an alkali metal salt of dichloro-s-triazinetrione or/and halogenated dimethylhydantoin. Lightcap reports that its composition inhibited the growth of algae in water and inactivated E. coli and Enterococcus faecium bacteria in water.

Randeri et al. (“Randeri”), U.S. Pat. No. 3,873,696 discloses that contact lenses can be effectively cleaned with a solution containing an oxygen-releasing salt such as a thiosulfate, a persulfate, or a peroxydisulfate. Randeri's preferred oxygen-releasing salt is potassium hydrogen peroxymonosulfate provided from a triple salt also containing potassium hydrogen sulfate (KHSO₄) and potassium sulfate (K₂SO₄). Randeri's solution normally contains a chloride-ion-releasing salt such as sodium chloride.

Potassium monopersulfate triple salt of the chemical formula 2KHSO₅.KHSO₄.K₂SO₄ is an implementation of the more general chemical composition (KHSO₅)ₓ(KHSO₄)_{y}(K₂SO₄)_{z} where x+y+z are variable mole (or molar) fractions whose sum is 1. The general composition (KHSO₅)ₓ(KHSO₄)_{y}(K₂SO₄)_{z} is referred to herein as “potassium monopersulfate triple salt composition” where the word “composition” distinguishes (KHSO₅)ₓ(KHSO₄)_{y}(K₂SO₄)_{z} from the specific formulation 2KHSO₅.KHSO₄.K₂SO₄. When mole fractions x, y, and z respectively are 0.5, 0.25, and 0.25, the potassium monopersulfate triple salt composition (KHSO₅)ₓ(KHSO₄)_{y}(K₂SO₄)_{z} becomes potassium monopersulfate triple salt of the formula 2KHSO₅.KHSO₄.K₂SO₄.

A molecule of potassium hydrogen peroxymonosulfate (KHSO₅) functions as an oxidizer by decomposing and “releasing” one of its oxygen atoms. The oxygen which can be so released from potassium hydrogen peroxymonosulfate is generally referred to as “active oxygen”. Pure potassium hydrogen peroxymonosulfate has an active oxygen content of approximately 10.5%.

The weight (mass) fractions of potassium hydrogen peroxymonosulfate (KHSO₅), potassium hydrogen sulfate (KHSO₄), and potassium sulfate (K₂SO₄) respectively are roughly 45%, 25%, and 30% in a common formulation of potassium monopersulfate triple salt composition (KHSO₅)ₓ(KHSO₄)_{y}(K₂SO₄)_{z}. This formulation has an active oxygen content of approximately 4.7%. The active oxygen content of potassium monopersulfate triple salt composition (KHSO₅)ₓ(KHSO₄)_{y}(K₂SO₄)_{z}, such as this formulation, is lower than the active oxygen content of potassium hydrogen peroxymonosulfate (KHSO₅) itself due to the presence of non-oxidizing components potassium hydrogen sulfate (KHSO₄) and potassium sulfate (K₂SO₄) in potassium monopersulfate triple salt composition. A small percentage of potassium persulfate (or potassium perdisulfate) of the chemical formula K₂S₂O₈ may be present in potassium peroxymonosulfate triple salt composition and in the specific potassium peroxymonosulfate triple salt. The potassium persulfate is generally undesirable because it reduces the active oxygen content, for example, to 4.5%. A higher active oxygen content is often desired.

The active oxygen content of potassium monopersulfate triple salt composition (KHSO₅)ₓ(KHSO₄)_{y}(K₂SO₄)_{z} decreases with time due to the decomposition of the potassium hydrogen peroxymonosulfate (KHSO₅) and the attendant release of some of the active oxygen. The decrease of active oxygen content with time is considerably greater for solutions containing potassium monopersulfate triple salt composition (KHSO₅)ₓ(KHSO₄)_{y}(K₂SO₄)_{z} than for dry solid potassium monopersulfate triple salt composition (KHSO₅)ₓ(KHSO₄)_{y}(K₂SO₄)_{z}. In particular, anhydrous solid potassium monopersulfate triple salt 2KHSO₅.KHSO₄.K₂SO₄ loses roughly 1% of its active oxygen content per month as reported in Tufano et al. (“Tufano), U.S. Pat. No. 6,818,142 B2. However, solutions of potassium monopersulfate triple salt 2KHSO₅.KHSO₄.K₂SO₄ lose 3-5% of their active oxygen content per month as indicated in Tufano and Durante et al. (Durante”), U.S. Pat. No. 7,442,323 B2. The solvent in the solutions referred to in Tufano and Durante presumably was high-purity water.

The loss of active oxygen content of potassium monopersulfate triple salt composition (KHSO₅)ₓ(KHSO₄)_{y}(K₂SO₄)_{z} with time depends on various factors including storage conditions, temperature, and material purity, including solution purity for (KHSO₅)ₓ(KHSO₄)_{y}(K₂SO₄)_{z} solutions. Regarding solution purity, (KHSO₅)ₓ(KHSO₄)_{y}(K₂SO₄)_{z} solutions made with normal water have greater loss in oxygen content with time than (KHSO₅)ₓ(KHSO₄)_{y}(K₂SO₄)_{z} solutions made with high-purity water because normal water contains a considerably higher content of metals than catalyze (promote) the decomposition of potassium hydrogen peroxymonosulfate (KHSO₅). In any event, the loss in active oxygen content significantly reduces the potency of potassium monopersulfate triple salt composition (KHSO₅)ₓ(KHSO₄)_{y}(K₂SO₄)_{z}, especially in solution form.

Potassium peroxymonosulfate triple salt composition (KHSO₅)ₓ(KHSO₄)_{y}(K₂SO₄)_{z}, including potassium peroxymonosulfate triple salt 2KHSO₅.KHSO₄.K₂SO₄ itself, can be manufactured in various ways. Martin, U.S. Pat. No. 7,090,820 B2, discloses a technique for manufacturing the potassium monopersulfate triple salt composition (KHSO₅)ₓ(KHSO₄)_{y}(K₂SO₄)_{z} with mole fractions x, y, and z adjusted to achieve an active oxygen content greater than 4.5% and with a reduced content of potassium persulfate. In a first embodiment, x is 0.43-0.64, y is 0.15-0.43, and z is 0.15-0.43. In a second embodiment, x is 0.46-0.64, y is 0.15-0.37, and z is 0.15-0.37.

Martin's process for manufacturing the potassium triple salt composition so that mole fractions x, y, and z fall into the ranges of the second embodiment entails adding a hydrogen peroxide solution containing at least 70% hydrogen peroxide by weight to a sulfuric acid solution containing at least 90% sulfuric acid by weight at a sulfuric-acid-to-hydrogen-peroxide ratio which is substoichiometric to produce a first Caro's acid solution containing peroxymonosulfuric acid (again, H₂SO₅) and hydrogen peroxide. The first Caro's acid solution is combined with oleum containing sulfuric acid and sulfur trioxide. The oleum reacts with water in the first Caro's acid solution to produce a second Caro's acid solution. An alkali potassium compound is added to the second Caro's acid solution to produce a partially neutralized solution containing potassium monopersulfate triple salt composition (KHSO₅)ₓ(KHSO₄)_{y}(K₂SO₄)_{z} in which x, y, and z have values in the ranges of Martin's second embodiment.

Looking now at the background art in total, bacterial, eukaryotic, prions, and viral pathogens cause many diseases to human. Success in treating these diseases varies widely. While treatments for some of these diseases are essentially fully successful, treatments for others are only partially successful or do not currently exist. In particular, treatments for allergic rhinitis, arthritis, bronchitis, hemorrhoids, urticaria, toothache, tinea pedis, acute viral nasopharyngitis, herpes simplex, dandruff, itching, bromhidrosis, and vaginitis are commonly weak or non-existent. Even when treatments are fully or partially successful, there are often adverse side effects to the treatments.

Sporkenbach's technique of using a peroxymonosulfuric acid salt, preferably potassium hydrogen peroxymonosulfate, to inactivate certain viruses on animate and inanimate surfaces is an advancement. However, Sporkenbach's viricidal technique is not useful in treating people to recover from the diseases caused by those viruses. It is desirable to have better techniques and medicinal products for treating people infected with diseases caused by bacterial, eukaryotic, prions, and viral pathogens and by non-pathogenic inflammation.

### GENERAL DISCLOSURE OF THE INVENTION

The present invention furnishes advanced products suitable for treating diseases and other debilitating medical conditions caused by bacterial, eukaryotic, prion, and viral pathogens, including fungal, spore, and parasitic pathogens, and by non-pathogenic inflammation. The products provided by the invention are also suitable for preventing debilitating medical conditions caused by bacterial, eukaryotic, prion, and viral pathogens and by non-pathogenic inflammation. In addition, the products provided by the invention can be used in other applications such as commercial and industrial applications.

More specifically, a product in a first aspect of the invention is a disintegrable composition for introduction into a liquid, normally water. The composition, normally consisting of solid material, is of such a structure that the composition gradually disintegrates upon introduction into the liquid for enabling particles of the composition to disperse into the liquid so that the mass of the composition is 10% to 90% of the initial mass value 1 hour to 100 days after the composition is introduced into the liquid. The gradual disintegration of the composition is generally referred to as metered release.

The disintegrable composition contains active material which is mainly responsible for the composition's advantageous properties. In one implementation of the disintegrable composition, the active material includes salt of peroxymonosulfuric acid, typically primarily potassium hydrogen peroxymonosulfate. In another implementation of the disintegrable composition, the active material includes inorganic halide and an oxidizing agent reactable in water with the inorganic halide to generate hypohalite ions.

The rate of disintegration can be adjusted to accommodate various situations. Relatively fast metered release of the disintegrable composition is, for example, useful in situations where a person ingests a piece of the disintegrable composition to treat a debilitating medical condition. The piece of the disintegrable composition then disintegrates in liquid, normally water, in the person's body so that the resultant liquid composition is administered internally to the person in a gradual manner.

Relatively slow metered release of the disintegrable composition is useful in situations in which the potency of the liquid form of the composition needs to be maintained suitably high for an extended period. A manufacturer of the liquid composition can employ slow metered release of the disintegrable composition to provide the liquid composition with extended shelf life at suitably high potency. A user of the liquid composition can utilize slow metered release of the disintegrable composition for maintaining the potency of the liquid composition suitably high for an extended period subsequent to combining the disintegrable composition with the liquid. Additionally, by providing the disintegrable composition as solid material, the disintegrable composition itself has a long shelf life.

A product in a second aspect of the invention is a composition formed with a carrier and active material dispersed largely throughout the carrier such that the composition is semiliquid. In one implementation of the semiliquid composition, the active material includes salt of peroxymonosulfuric acid or/and reaction product of the salt of peroxymonosulfuric acid and other material of the composition. The salt of peroxymonosulfuric acid is again typically primarily potassium hydrogen peroxymonosulfate. In another implementation of the semiliquid composition, the active material includes (a) inorganic halide or/and reaction product of the inorganic halide and other material of the composition and (b) an oxidizing agent or/and reaction product of the oxidizing agent and other material of the composition. The oxidizing agent is again reactable in water with the inorganic halide to generate hypohalite ions.

The semiliquid composition in the first aspect of the invention normally has a dynamic viscosity of at least 5 Pa-s at 25° C. The dynamic viscosity of the composition is normally no more than 5,000 Pa-s at 25° C. By providing the composition in semiliquid form, particularly with a dynamic viscosity of 5 to 5,000 Pa-s at 25° C., the shelf life of the composition is expected to be extended while simultaneously enabling the composition to be readily administered.

A product in a third aspect of the invention is a container assembly consisting at least partially of a composition and a container that holds the composition. The components of the composition, normally a liquid, include a carrier and active material dispersed largely throughout the carrier. In one implementation of the container assembly, the active material includes salt of peroxymonosulfuric acid or/and reaction product of the salt of peroxymonosulfuric acid and other material of the composition. The salt of peroxymonosulfuric acid is once again typically primarily potassium hydrogen peroxymonosulfate. In another implementation of the container assembly, the active material includes (a) inorganic halide or/and reaction product of the inorganic halide and other material of the composition and (b) an oxidizing agent or/and reaction product of the oxidizing agent and other material of the composition. The oxidizing agent is once again reactable in water with the inorganic halide to generate hypohalite ions.

The container blocks transmission of visible light, and typically also other radiation such as ultraviolet (“UV”) radiation, incident on the container from outside the container in one embodiment of the product in the third aspect of the invention. This prevents visible light, and typically also such other radiation, from degrading the composition, thereby enabling it to have extended shelf life at suitably high potency. In another embodiment of this product, the composition is subjected to an average pressure of more than 1 atm, preferably at least 2 atm, inside the container. The elevated pressure inside the container assists in dispensing the product from the container while preventing material, such as air, outside the container from contaminating the composition and causing it to degrade. The shelf life at suitably high potency is again extended.

The product in the third aspect of the invention normally includes a dispenser hermetically attached to the container for controllably dispensing the composition from the container. In a further embodiment of the product, the dispenser prevents material outside the container and the dispenser from entering the container through the dispenser. This similarly extends the shelf life by preventing material, such as air, outside the container from contaminating the composition and causing it to degrade. The features of these three embodiments may be variously combined in other embodiments of this product.

A product in a fourth aspect of the invention is a multiple-container assembly consisting at least partially of a first container, a second container, and a combining element attached to or integral with at least one of the containers. The first container contains a liquid carrier. The second container contains a primary composition containing active material. The combining element enables matter of the primary composition and matter of the carrier to be combined to form a further composition.

The active material includes salt of peroxymonosulfuric acid, again typically primarily potassium hydrogen peroxymonosulfate, in one implementation of the double-container assembly. In another implementation of the multiple-container assembly, the active material includes inorganic halide and an oxidizing agent reactable in water with the inorganic halide to generate hypohalite ions.

All the material of the primary composition is normally solid material. The further liquid composition created by combining matter of the primary composition and matter of the carrier need not be formed until shortly before the further liquid composition is administered to a person. As a result, the multiple-container assembly has a long shelf life.

A product in a fifth aspect of the invention is a composition containing a plurality of solid particles consisting of support material and active material. Because the particles are solid, the particle-containing composition has a long shelf life.

The active material includes salt of peroxymonosulfuric acid, once again typically primarily potassium hydrogen peroxymonosulfate, in one implementation of the particle-containing composition. The salt of peroxymonosulfuric acid is present in the particles at an average mass percentage of no more than 10%. The support material is normally non-reactive with the salt of peroxymonosulfuric acid and with reaction product of the salt of peroxymonosulfuric acid and any other material of the composition when it is dry or combined with water.

In another implementation of the particle-containing composition, the active material includes inorganic halide and an oxidizing agent reactable in water with the inorganic halide to generate hypohalite ions. The oxidizing agent is present in the particles at an average mass percentage of no more than 10%. The support material is normally non-reactive with the oxidizing agent and with reaction product of the oxidizing agent and any other material of the composition when it is dry or combined with water.

The particles containing either implementation of the particle-containing composition are normally so small that they form a powder. In particular, the average diameter of the particles is normally no more than 500 μm.

A further composition is formed by combining the particles containing either implementation of the particle-containing composition with a liquid carrier, normally water. The liquid carrier promotes chemical reactions involving the active material. However, the particle support material and the liquid carrier can readily be chosen so that the support material is chemically non-reactive in the presence of the carrier. As a result, comparatively fewer chemical reactions involving the active material are expected to occur. This, in turn, is expected to enable the further composition to have suitably high potency for increased time subsequent to combining the particles with the liquid carrier.

A product in a sixth aspect of the invention is a composition containing a liquid carrier, active material dispersed largely throughout the carrier, and an inhibitor dispersed largely throughout the carrier. The active material includes an oxidizing agent or/and reaction product of the oxidizing agent and other material of the composition. The oxidizing agent contains active oxygen that consists of chemically readily transferable oxygen atoms. The inhibitor inhibits the composition from losing active oxygen. As a result, the potency of the inhibitor-containing composition is maintained at a suitably high level for extended time so that the composition has increased shelf life.

The oxidizing agent in one implementation of the inhibitor-containing composition is salt of peroxymonosulfuric acid, normally primarily potassium hydrogen peroxymonosulfate. In another implementation of the inhibitor-containing composition, the active material includes inorganic halide in addition to the oxidizing agent. With the liquid carrier including water in this implementation, the oxidizing agent is reactable in water with the inorganic halide to generate hypohalite ions.

In short, the invention furnishes a variety of products having extended shelf life. The products of the invention are suitable for treating diseases and other debilitating medical conditions caused by bacterial, eukaryotic, prion, and viral pathogens, including fungal, spore, and parasitic pathogens, and by non-pathogenic inflammation and for preventing debilitating medical conditions caused by bacterial, eukaryotic, prion, and viral pathogens and by non-pathogenic inflammation. The products of the invention can also be used in other applications. The invention thereby provides a large advance over the prior art.

### BRIEF DESCRIPTION OF THE DRAWINGS

FIG. 1 is a side cross-sectional view of a container assembly in accordance with the invention for dispensing a medicinal drug.

FIGS. 2a and 2b are side cross-sectional views of a typical embodiment of the dispenser in the container assembly of FIG. 1 respectively in the non-actuated and actuated conditions.

FIG. 3 is a side cross-sectional/schematic view of a double-container assembly in accordance with the invention for dispensing a medicinal drug.

FIG. 4 is a side cross-sectional view of a powder of particles that contain a medicinal drug in accordance with the invention.

Like reference symbols are used in the drawings and in the description of the preferred embodiments to represent the same, or very similar, item or items.

### DESCRIPTION OF THE PREFERRED EMBODIMENTS

Persons afflicted with diseases and other debilitating medical conditions caused by bacterial, eukaryotic, prion, and viral pathogens, including fungal, spore-caused, and parasitic infections, and by non-pathogenic-caused inflammation are treated with a medicinal drug formed at least partially with salt, i.e., one or more individual salts, of peroxymonosulfuric acid (H₂SO₅). Such a salt is generally referred to as a peroxymonosulfate. The medicinal drug formed at least partially with peroxymonosulfate is referred to herein as being of type I. The medicinal drug of type I can also be administered to persons for preventing them from contracting debilitating medical conditions caused by bacterial, eukaryotic, prion, and viral pathogens, including fungal, spore-caused, and parasitic infections, and by non-pathogenic-caused inflammation.

Peroxymonosulfates are generally strong oxidizing agents, i.e., they readily provide (or release) oxygen under certain conditions at standard temperature (20-25° C.) and standard pressure (760 torr). In particular, oxygen is readily released by a peroxymonosulfate when it is present in water, typically in aqueous solution. The active oxygen content R_{AO}, i.e., fraction or percentage of readily providable oxygen, of an oxidizing agent such as a peroxymonosulfate is given generally as:

R_{AO}=m_{AO}/m_{OXAG}=W_{O}N/W_{OXAG} (1)

where m_{AO} is the mass of the active oxygen in the oxidizing agent, m_{OXAG} is the mass of the oxidizing agent, W_{O} is the molecular weight of oxygen, N is the number of moles of active oxygen in a mole of the oxidizing agent, and W_{OXAG} is the molecular weight of the oxidizing agent.

Oxygen's molecular weight W_{O} is approximately 16.00. Accordingly, active oxygen content R_{AO} is more particularly given as:

R_{AO}≈16.00 N/W_{OXAG} (2)

The oxidizing capability of the peroxymonosulfate used in forming the medicinal drug of type I is believed to be a factor in the drug's effectiveness in combating bacterial, eukaryotic, prion, and viral pathogens that attack humans and in causing non-pathogenic-caused inflammation to be reduced in humans. Eq. 2 thus provides an estimate of the oxidizing capability of the peroxymonosulfate used in forming the drug of type I.

The peroxymonosulfates include alkali metal salts, alkaline earth metal salts, and ammonium (group) salts of peroxymonosulfuric acid. Such a peroxymonosulfate is chemically representable as MᵢHⱼ(SO₅)ₖ where M is an alkali metal in Group 1a of the Periodic Table, an alkaline earth metal in Group 1b of the Periodic Table, or an ammonium group and where i, j, and k are integers. Integers i, j, and k satisfy the relationship ni+j equals 2k where n is an integer equal to 1 for an alkali metal or an ammonium group and equal to 2 for an alkaline earth metal. Integer j can be 0 such that hydrogen is absent in the Hⱼ term of MᵢHⱼ(SO₅)ₖ.

The alkali metal salts of peroxymonosulfuric acid consist of its alkali metal hydrogen salts and its dialkali metal salts. For primary alkali metals lithium, sodium, and potassium, the alkali metal salts of peroxymonosulfuric acid are lithium hydrogen peroxymonosulfate (LiHSO₅), dilithium peroxymonosulfate (Li₂SO₅), sodium hydrogen peroxymonosulfate (NaHSO₅), disodium peroxymonosulfate (Na₂SO₅), potassium hydrogen peroxymonosulfate (KHSO₅), and dipotassium peroxymonosulfate (K₂SO₅). Similar to potassium hydrogen peroxymonosulfate often referred to as potassium peroxymonosulfate, lithium hydrogen peroxymonosulfate and sodium hydrogen peroxymonosulfate may respectively be referred to simply as lithium peroxymonosulfate and sodium peroxymonosulfate.

Rubidium and cesium are additional alkali metals. To the extent manufacturable without being toxic, the alkali metal salts of peroxymonosulfuric acid further include rubidium hydrogen peroxymonosulfate (RbHSO₅), dirubidium peroxymonosulfate (Rb₂SO₅), cesium hydrogen peroxymonosulfate (CsHSO₅), and dicesium peroxymonosulfate (Cs₂SO₅). Rubidium hydrogen peroxymonosulfate and cesium hydrogen peroxymonosulfate may respectively be referred to simply as rubidium peroxymonosulfate and cesium peroxymonosulfate.

The alkaline earth metal salts of peroxymonosulfuric acid consist of its alkaline earth metal salts and its alkaline earth metal hydrogen salts. For primary alkaline earth metals magnesium and calcium, the alkaline earth metal salts of peroxymonosulfuric acid are magnesium peroxymonosulfate (MgSO₅), magnesium dihydrogen diperoxymonosulfate (MgH₂(SO₅)₂), calcium peroxymonosulfate (CaSO₅), and calcium dihydrogen diperoxymonosulfate (CaH₂(SO₅)₂).

Beryllium, barium, and strontium are additional alkaline earth metals. To the extent manufacturable without being toxic, the alkaline earth metal salts of peroxymonosulfuric acid further include beryllium peroxymonosulfate (BeSO₅), beryllium dihydrogen diperoxymonosulfate (BeH₂(SO₅)₂), barium peroxymonosulfate (BaSO₅), barium dihydrogen diperoxymonosulfate (BaH₂(SO₅)₂), strontium peroxymonosulfate (SrSO₅), and strontium dihydrogen diperoxymonosulfate (SrH₂(SO₅)₂).

An ammonium salt of peroxymonosulfuric acid is chemically representable as NRₐR_{b}RₑR_{d}HSO₅ or as NRₐR_{b}R_{c}R_{d}NRₑR_{f}R_{g}RₕSO₅ where each of Rₐ, R_{b}, R_{c}, R_{d}, Rₑ, R_{f}, R_{g}, and Rₕ is variously hydrogen or a hydrocarbon group such as an alkyl, cycloalkyl, aryl, or aralkyl group. For the NRₐR_{b}R_{c}R_{d}NRₑR_{f}R_{g}RₕSO₅ ammonium peroxymonosulfates, Rₑ, R_{f}, R_{g}, and Rₕ are typically respectively the same as Rₐ, R_{b}, R_{c}, and R_{d}. The non-carbon ammonium salts of peroxymonosulfuric acid are ammonium hydrogen peroxymonosulfate (NH₄HSO₅) and diammonium peroxymonosulfate ((NH₄)₂ SO₅). Ammonium hydrogen peroxymonosulfate may be referred to simply as ammonium peroxymonosulfate.

Peroxymonosulfuric acid may also form salts with metals in parts of the Periodic Table other than the alkali metals of Group 1a and the alkaline earth metals of Group 1b. For instance, peroxymonosulfuric acid may form salts with metals, e.g., zinc, in Group 2b of the Periodic Table. To the extent manufacturable, the Group 2b salts of peroxymonosulfuric acid include zinc peroxymonosulfate (ZnSO₅) and zinc dihydrogen diperoxymonosulfate (ZnH₂(SO₅)₂).

The salt of peroxymonosulfuric acid used in forming the medicinal drug of type I is preferably potassium hydrogen peroxymonosulfate (KHSO₅). In a molecule of potassium hydrogen peroxymonosulfate, a pair of oxygen atoms singly bonded to each other are situated between the sulfur and hydrogen atoms. The single oxygen-oxygen bond readily breaks under certain conditions, e.g., when the molecule of potassium hydrogen peroxymonosulfate is dissolved in a suitable solvent such as water, to release one of the oxygen atoms involved in the single oxygen-oxygen bond. The molecular weight W_{KHSO5} of potassium hydrogen peroxymonosulfate is approximately 152.17. Potassium hydrogen peroxymonosulfate has one mole of active oxygen per mole of potassium hydrogen peroxymonosulfate. Utilizing Eq. 2 given above, active oxygen content R_{AO} of pure potassium hydrogen peroxymonosulfate is approximately 10.5%.

The potassium hydrogen peroxymonosulfate used in forming the medicinal drug of type I is normally provided as a component of a multiple salt, preferably potassium monopersulfate triple salt composition (KHSO₅)ₓ(KHSO₄)_{y}(K₂SO₄)_{z} for which the sum of mole fractions x, y, and z equals 1. Potassium hydrogen sulfate (KHSO₄) and potassium sulfate (K₂SO₄), again sometimes referred to as dipotassium sulfate, are physically bonded to potassium hydrogen peroxymonosulfate in potassium monopersulfate triple salt composition.

Neither potassium hydrogen sulfate nor potassium sulfate readily releases oxygen under the same conditions at standard temperature and pressure for which potassium hydrogen peroxymonosulfate readily releases oxygen. The overall oxidizing agent formed with potassium monopersulfate triple salt composition normally used in forming the medicinal drug of type I thereby consists of (a) active oxygen-releasing material formed by potassium hydrogen peroxymonosulfate and (b) other material, referred to here as inactive material, consisting of potassium hydrogen sulfate and potassium sulfate.

The mass fraction F_{Mp} of a component Cₚ of a general product having n components C₁, . . . C_{q}, . . . Cₙ, each being of a molecular weight W_{q} and of a mole fraction F_{Mq} in the product, is given as:

$${F}_{m p} = {W}_{p} {F}_{Mp} / {\sum }_{q = 1}^{n} {W}_{q} {F}_{Mq} \qquad ( 3 )$$

where q is an integer varying from 1 to n and where the sum of mole fractions F_{M1}, . . . F_{Mq}, . . . F_{Mn} equals 1. The molecular weights W_{K2SO5}, W_{KHSO4}, and W_{K2SO4} of potassium hydrogen peroxymonosulfate, potassium hydrogen sulfate, and potassium sulfate respectively are 152.17, 136.17, and 174.26 (to two significant digits beyond the decimal point). Letting F_{mK2SO5}, F_{mKHSO4}, and F_{mK2SO4} represent the respective mass fractions of potassium hydrogen peroxymonosulfate, potassium hydrogen sulfate, and potassium sulfate in potassium monopersulfate triple salt composition (KHSO₅)ₓ(KHSO₄)_{y}(K₂SO₄)_{z}, mass fractions F_{mK2SO5}, F_{mKHSO4}, and F_{mK2SO4} are respectively given in terms of mole fraction x of potassium hydrogen peroxymonosulfate, mole fraction y of potassium hydrogen sulfate, and mole fraction z of potassium sulfate as:

$$\begin{matrix}\begin{matrix}{F}_{mKHSO 5} = {W}_{KHSO 5} x / ( {W}_{KHSO 5} x + {W}_{KHSO 4} y + {W}_{K 2 SO 4} z ) \\ \approx 152.17 x / ( 152.17 x + 136.17 y + 174.26 z )\end{matrix} & ( 4 ) \\ \begin{matrix}{F}_{mKHSO 4} = {W}_{KHSO 4} x / ( {W}_{KHSO 5} x + {W}_{KHSO 4} y + {W}_{K 2 SO 4} z ) \\ \approx 136.17 x / ( 152.17 x + 136.17 y + 174.26 z )\end{matrix} & ( 5 ) \\ \begin{matrix}{F}_{mK 2 SO 4} = {W}_{K 2 SO 4} x / ( {W}_{KHSO 5} x + {W}_{KHSO 4} y + {W}_{K 2 SO 4} z ) \\ \approx 174.26 x / ( 152.17 x + 136.17 y + 174.26 z )\end{matrix} & ( 6 )\end{matrix}$$

Potassium monopersulfate triple salt composition is implemented with potassium monopersulfate triple salt (2KHSO₅.KHSO₄.K₂SO₄) in one formulation used in forming the medicinal drug of type I. Inasmuch as mole fractions x, y, and z respectively are 0.5, 0.25, and 0.25 in this formulation, use of Eqs. 4-6 yields the following values for mass fractions F_{mK2SO5}, F_{mKHSO4}, and F_{mK2SO4} in this formulation: (a) mass fraction F_{mK2SO5} of potassium hydrogen peroxymonosulfate is approximately 50%, (b) mass fraction F_{mKHSO4} of potassium hydrogen sulfate is approximately 22%, and (c) mass fraction F_{mK2SO4} of potassium sulfate is approximately 28%.

The active oxygen content R_{AO}, i.e., fraction or percentage of active oxygen, in potassium monopersulfate triple salt composition is given in terms of mole fractions x, y, and z by the relationship:

$$\begin{matrix}{R}_{AO} = {W}_{O} x / ( {W}_{KHSO 5} x + {W}_{KHSO 4} y + {W}_{K 2 SO 4} z ) \\ \approx 16.00 x / ( 152.17 x + 136.17 y + 174.26 z )\end{matrix} \qquad ( 7 )$$

where W_{O} is again the molecular weight of oxygen. For the preceding formulation in which mole fractions x, y, and z respectively are 0.5, 0.25, and 0.25, active oxygen content R_{AO} is approximately 5.2%.

The mole fraction F_{Mp} of component Cₚ in a general product is, in turn, given as:

$${F}_{Mp} = ( \frac{{F}_{m p}}{{W}_{p}} ) / {\sum }_{q = 1}^{n} ( \frac{{F}_{mq}}{{W}_{q}} ) \qquad ( 8 )$$

Accordingly, mole fractions x, y, and z respectively of potassium hydrogen peroxymonosulfate, potassium hydrogen sulfate, and potassium sulfate in potassium monopersulfate triple salt composition (KHSO₅)ₓ(KHSO₄)_{y}(K₂SO₄)_{z} are respectively given in terms of mass fractions F_{mK2SO5}, F_{mKHSO4}, and F_{mK2SO4} as:

$$\begin{matrix}\begin{matrix}x = ( \frac{{F}_{mKHSO 5}}{{W}_{KHSO 5}} ) / ( \frac{{F}_{mKHSO 5}}{{W}_{KHSO 5}} + \frac{{F}_{mKHSO 4}}{{W}_{KHSO 4}} + \frac{{F}_{mK 2 SO 4}}{{W}_{K 2 SO 4}} ) \\ \approx ( \frac{{F}_{mKHSO 5}}{152.17} ) / ( \frac{{F}_{mKHSO 5}}{152.17} + \frac{{F}_{mKHSO 4}}{136.17} + \frac{{F}_{mK 2 SO 4}}{174.26} )\end{matrix} & ( 9 ) \\ \begin{matrix}y = ( \frac{{F}_{mKHSO 4}}{{W}_{KHSO 4}} ) / ( \frac{{F}_{mKHSO 5}}{{W}_{KHSO 5}} + \frac{{F}_{mKHSO 4}}{{W}_{KHSO 4}} + \frac{{F}_{mK 2 SO 4}}{{W}_{K 2 SO 4}} ) \\ \approx ( \frac{{F}_{mKHSO 4}}{136.17} ) / ( \frac{{F}_{mKHSO 5}}{152.17} + \frac{{F}_{mKHSO 4}}{136.17} + \frac{{F}_{mK 2 SO 4}}{174.26} )\end{matrix} & ( 10 ) \\ \begin{matrix}z = ( \frac{{F}_{mK 2 SO 4}}{{W}_{K 2 SO 4}} ) / ( \frac{{F}_{mKHSO 5}}{{W}_{KHSO 5}} + \frac{{F}_{mKHSO 4}}{{W}_{KHSO 4}} + \frac{{F}_{mK 2 SO 4}}{{W}_{K 2 SO 4}} ) \\ \approx ( \frac{{F}_{mK 2 SO 4}}{174.26} ) / ( \frac{{F}_{mKHSO 5}}{152.17} + \frac{{F}_{mKHSO 4}}{136.17} + \frac{{F}_{mK 2 SO 4}}{174.26} )\end{matrix} & ( 11 )\end{matrix}$$

Mass fractions F_{mKHSO5}, F_{mKHSO4}, and F_{mK2SO4} respectively are approximately 45%, 25%, and 30% in another formulation of potassium monopersulfate triple salt composition used in forming the medicinal drug of type I. Use of Eqs. 9-11 yields the following values for mole fractions x, y, and z in this second formulation: (a) mole fraction x of potassium hydrogen peroxymonosulfate is approximately 46%, (b) mole fraction y of potassium hydrogen sulfate is approximately 28%, and (c) mole fraction y of potassium sulfate is approximately 26%. The second formulation of the potassium monopersulfate triple salt composition is then approximately given as (KHSO₅)_{0.46}(KHSO₄)_{0.28}(K₂SO₄)_{0.26} or approximately equivalently as 1.7KHSO₅.1.1KHSO₄.K₂SO₄.

Active oxygen content R_{AO} is given in terms of mass fraction F_{mKHSO5} as:

$$\begin{matrix}{R}_{AO} = ( {W}_{O} / {W}_{KHSO 5} ) {F}_{KHSO 5} \\ \approx ( 16.00 / 152.17 ) {F}_{KHSO 5} \\ \approx 0.1051 {F}_{KHSO 5}\end{matrix} \qquad ( 12 )$$

For the second formulation of potassium monopersulfate triple salt composition in which mass fraction F_{mKHSO5} is approximately 45%, active oxygen content R_{AO} is approximately 4.7%.

The medicinal drug of Type I may be formed with a small percentage, normally no more than a few percent by mass, typically no more than 1% by mass, of one or more other potassium-sulfur-oxygen salts, such as potassium persulfate (K₂S₂O₈), present as impurity in the potassium monopersulfate triple salt composition. Although this might cause the resultant composition of potassium hydrogen peroxymonosulfate, potassium hydrogen sulfate, potassium sulfate, and each other potassium-sulfur-oxygen salt to strictly be a multiple salt of at least four potassium-sulfur-oxygen salts, the resultant composition is substantially potassium monopersulfate triple salt composition because the impurity potassium-sulfur-oxygen salt constitutes only a small percentage by mass of the resultant composition. Furthermore, the potassium hydrogen peroxymonosulfate, potassium hydrogen sulfate, and potassium sulfate used in forming the resultant composition still constitute potassium monopersulfate triple salt composition.

The medicinal drug of type I may be formed with one or more components in addition to salt of peroxymonosulfuric acid and, when the salt of peroxymonosulfuric acid consists of potassium hydrogen peroxymonosulfate provided as a component of potassium monopersulfate triple salt composition, one or more components in addition to potassium hydrogen sulfate, potassium sulfate, and any impurity in the potassium monopersulfate triple salt composition. The components used to form the drug of type I are preferably all water soluble such that the drug of type I is water soluble. The drug of type I is, as discussed further below, typically provided in a therapeutically inactive pharmaceutically acceptable carrier, often water in which the components used to form the drug are dissolved. One or more of the components used to form the drug of type I may, nonetheless, be non-soluble in water. In that case, each non-water-soluble component is normally in liquid form or colloidably suspendable in water.

Formulations of potassium peroxymonosulfate triple salt composition suitable for use in forming the medicinal drug of type I can be manufactured in various ways. It is typically desirable that a (KHSO₅)ₓ(KHSO₄)_{y}(K₂SO₄)_{z} formulation used in forming the drug of type I have a higher active oxygen content, and a lower potassium persulfate impurity content, than the second formulation of potassium monopersulfate triple salt composition in which mass fraction F_{mKHSO5} is approximately 45%.

With reference to Martin, cited above, a suitable process for manufacturing the potassium triple salt composition entails first adding a hydrogen peroxide (H₂O₂) solution containing at least 70% hydrogen peroxide by mass to a sulfuric acid (H₂SO₄) solution containing at least 90% sulfuric acid by mass at a substoichiometric sulfuric-acid-to-hydrogen-peroxide ratio to produce a first Caro's acid solution containing peroxymonosulfuric acid and hydrogen peroxide. The first Caro's acid solution is then combined with oleum containing sulfuric acid and sulfur trioxide (SO₃). The oleum reacts with water in the first Caro's acid solution to produce a second Caro's acid solution. The temperature is maintained below 30°, normally below 20° C., during the preceding steps.

A potassium compound is added to the second Caro's acid solution to produce a partially neutralized solution containing potassium monopersulfate triple salt composition (KHSO₅)ₓ(KHSO₄)_{y}(K₂SO₄)_{z}. The partially neutralized solution is concentrated to form a slurry of the potassium monopersulfate triple salt composition at a desired specific gravity, e.g., 1.55-1.65. The slurry is typically formed by mixing in a vacuum evaporator at a temperature of no more than 35° C. The slurry is separated into mother liquor and solids of which the solids contain the monopersulfate triple salt composition. The solids are dried at a temperature of no more than 90° C., preferably no more than 70° C. Further details on this process are presented in Martin, the contents of which are incorporated by reference herein.

By suitably controlling the process conditions, mole fractions x, y, and z of the potassium monopersulfate triple salt composition (KHSO₅)ₓ(KHSO₄)_{y}(K₂SO₄)_{z} fall into the following ranges. Broadly, x is 0.43-0.64, y is 0.15-0.43, and z is 0.15-0.43. More narrowly, x is 0.46-0.64, y is 0.15-0.37, and z is 0.15-0.37.

The specific values of mole fractions x, y, and z within the preceding ranges are often selected so that active oxygen content R_{AO} of the resultant formulation of the potassium monopersulfate triple salt composition is greater than the approximate 4.7% active oxygen content of the second formulation of potassium monopersulfate triple salt composition in which mass fraction F_{mKHSO5} of potassium hydrogen peroxymonosulfate is approximately 45% and in which corresponding mole fraction x of potassium hydrogen peroxymonosulfate is approximately 46%. For instance, mole fractions x, y, and z are typically chosen active oxygen content R_{AO} of the formulation of the potassium monopersulfate triple salt composition is greater than 4.9%. At the same time, the amount of potassium persulfate in the formulation of the potassium monopersulfate triple salt composition is less than 0.5% by mass.

Increasing mole fraction x of potassium hydrogen peroxymonosulfate in potassium monopersulfate triple salt composition (KHSO₅)ₓ(KHSO₄)_{y}(K₂SO₄)_{z} generally causes active oxygen content R_{AO} to increase. For example, an R_{AO} value of 5.2%, and thus greater than 4.7%, is achieved with the first-mentioned formulation in which mole fractions x, y, and z respectively are 0.5, 0.25, and 0.25. Active oxygen content R_{AO} of potassium monopersulfate triple salt composition can be made greater than 6% by choosing mole fraction x of potassium hydrogen peroxymonosulfate to be at, or close to, the upper limit of 0.64 in the two sets of mole fraction ranges given above. As another example, use of Eq. 7 yields an R_{AO} value of approximately 6.7% when mole fraction x is 0.64 and mole fractions y and z both are 0.18.

As described in Martin, the foregoing process can be modified in various ways. For instance, a suprastoichiometric sulfuric-acid-to-hydrogen-peroxide ratio can be used instead of a substoichiometric sulfuric-acid-to-hydrogen-peroxide ratio. In that case, mole fractions x, y, and z of the potassium monopersulfate triple salt composition (KHSO₅)ₓ(KHSO₄)_{y}(K₂SO₄)_{z} fall into the further narrowed range set in which x is 0.53-0.64, y is 0.15-0.37, and z is 0.15-0.37.

When mole fraction x equals 0.53 at the lower end of the mole fraction range for potassium hydrogen peroxymonosulfate in this third set of mole fraction ranges, use of Eq. 7 yields an R_{AO} value of approximately of 5.5% when mole fractions y and z both are 0.235. Since the upper limit of mole fraction x is 0.64 in this third set of mole fraction ranges, active oxygen content R_{AO} of potassium monopersulfate triple salt composition can readily be chosen to be 5.5-6.8% by choosing mole fractions x, y, and z to appropriately fall into this third set of mole fraction ranges.

People afflicted with diseases and other debilitating medical conditions caused by bacterial, eukaryotic, prion, and viral pathogens, again including fungal, spore-caused, and parasitic infections, and by non-pathogenic-caused inflammation are treated with a medicinal drug formed at least partially with water-soluble inorganic halide and an oxidizing agent reactable in water, typically aqueous solution, with the halide to generate hypohalite ions. The medicinal drug is also normally formed at least partially with (anhydrous) alkali metal phosphate and sulfamic acid. The chemical formula for sulfamic acid, alternatively known as amidosulfonic acid, amidosulfuric acid, aminosulfonic acid, and sulfamidic acid, is H₃NSO₃. The medicinal drug formed at least partially with the preceding four components is referred to herein as being of type II. The medicinal drug of type II can also be administered to persons for preventing them from contracting debilitating medical conditions caused by bacterial, eukaryotic, prion, and viral pathogens, including fungal, spore-caused, and parasitic infections, and by non-pathogenic-caused inflammation.

The oxidizing agent used in forming the medicinal drug of type II contains active oxygen-releasing material that readily releases oxygen under certain conditions at standard temperature and standard pressure. More particularly, oxygen is readily released by the active-oxygen-releasing material when it is present in water, typically in aqueous solution. The oxidizing agent typically may include additional oxygen-containing material, referred to here as inactive material, which does not readily release oxygen under the same conditions at standard temperature and pressure for which the active oxygen-releasing material readily releases oxygen.

The ratio of the mole fraction F_{MHI} of the inorganic halide to the mole fraction F_{MAORM} of the active oxygen-releasing material in the medicinal drug of type II is normally 0.01-4, preferably 0.02-2, more preferably 0.03-1, even more preferably 0.04-0.5, typically 0.06-0.18. The ratio of the mole fraction F_{MAMP} of the alkali metal phosphate to mole fraction F_{MAORM} of the active oxygen-releasing material in the drug of type II is normally 0.04-40, preferably 0.1-5, more preferably 0.2-1, typically 0.4 The ratio of the mole fraction F_{MSA} of sulfamic acid to mole fraction F_{MAORM} of the active oxygen-releasing material in the drug of type II is normally 0.03-30, preferably 0.1-10, more preferably 0.2-1, typically 0.35-0.7.

The inorganic halide is normally sodium chloride (NaCl), an alkali metal halide. The inorganic halide can alternatively or additionally include one or more other alkali metal halides, one or more alkaline earth metal halides, or/and one or more ammonium halides. In particular, the inorganic halide can alternatively or additionally include one or more of lithium fluoride (LiF), sodium fluoride (NaF), potassium fluoride (KF), rubidium fluoride (RbF), cesium fluoride (CsF), beryllium fluoride (BeF₂), magnesium fluoride (MgF₂), calcium fluoride (CaF₂), strontium fluoride (SrF₂), barium fluoride (BaF₂), ammonium fluoride (NH₄F), lithium chloride (LiCl), potassium chloride (KCl), rubidium chloride (RbCl), cesium chloride (CsCl), beryllium chloride (BeCl₂), magnesium chloride (MgCl₂), calcium chloride (CaCl₂), strontium chloride (SrCl₂), barium chloride (BaCl₂), ammonium chloride (NH₄Cl), lithium bromide (LiBr), sodium bromide (NaBr), potassium bromide (KBr), rubidium bromide (RbBr), cesium bromide (CsBr), beryllium bromide (BeBr₂), magnesium bromide (MgBr₂), calcium bromide (CaBr₂), strontium bromide (SrBr₂), barium bromide (BaBr₂), ammonium bromide (NH₄Br), lithium iodide (LiI), sodium iodide (NaI), potassium iodide (KI), rubidium iodide (RbI), cesium iodide (CsI), beryllium iodide (BeI₂), magnesium iodide (MO2), calcium iodide (CaI₂), strontium iodide (SrI₂), barium iodide (BaI₂), and ammonium iodide (NH₄I) provided that such other halide does not react with the alkali metal phosphate to form an insoluble salt.

The alkali metal phosphate is normally one or more of sodium metaphosphate ((NaPO₃)ₙ), monosodium phosphate (NaH₂PO₄), disodium phosphate (Na₂HPO₄), trisodium phosphate (Na₃PO₄), tetrasodium pyrophosphate (Na₄P₂O₇), potassium metaphosphate ((KPO₃)ₙ), monopotassium phosphate (KH₂PO₄), dipotassium phosphate (K₂HPO₄), tripotassium phosphate (K₃PO₄), and tetrapotassium pyrophosphate (K₄P₂O₇). For each of sodium metaphosphate and potassium metaphosphate, n is a plural integer, i.e., n is 2 or more. Sodium metaphosphate can, for example, be implemented with sodium hexametaphosphate ((NaPO₃)₆) or/and sodium trimetaphosphate ((NaPO₃)₃). Potassium metaphosphate can similarly be implemented with potassium hexametaphosphate ((KPO₃)₆) or/and potassium trimetaphosphate ((KPO₃)₃).

The material used to form the medicinal drug of type II may include a non-reducing organic acid, normally malic acid (HO₂CCH₂CHOHCO₂H) or succinic acid (HO₂CCH₂CH₂CO₂H). The ratio of the mole fraction F_{MNROA} of the non-reducing organic acid to mole fraction F_{MAORM} of the active oxygen-releasing material in the drug of type II is normally 0.01-50, preferably 0.05-5, more preferably 0.1-1, typically 0.25.

The material used to form medicinal drug of type II may also include a surfactant, typically sodium dodecylbenzene sulfonate (NaC₁₈H₂₉SO₃). Other candidates for the surfactant are lauryl ether sulfates, ethylene oxide/propylene oxide alkyl phenol condensates, polyglycol ethers of fatty alcohols, fatty acid ethylene oxide condensates, polyglycol ethers of aklyn phenols, and fatty alcohol ethoxylates. The ratio of the mole fraction F_{MSF} of the surfactant to mole fraction F_{MAORM} of the active oxygen-releasing material in the drug of type II is normally 0.03-30, preferably 0.05-10, more preferably 0.1-10, typically 0.3.

The medicinal drug of type II is, as discussed further below, often provided in a therapeutically inactive pharmaceutically acceptable liquid carrier, normally water. Each of the components, including the oxidizing agent, of the material used to form the drug of type II is then soluble, e.g., water soluble, in the carrier, colloidably suspendable in the carrier, or in liquid form so as to be miscible or emulsible with the carrier. The components used to form the drug of type II are preferably all water soluble such that the drug of type II is water soluble.

The active-oxygen-releasing material in the oxidizing agent for the medicinal drug of type II normally includes persulfate or/and peroxyphthalate. Persulfate candidates for the active-oxygen-releasing material include peroxymonosulfates (having SO₅⁻² groups) and peroxydisulfates (having S₂O₈⁻² groups). The active-oxygen-releasing material preferably includes peroxymonosulfate, i.e., salt of peroxymonosulfuric acid, such as alkali metal salt, alkaline-earth metal salt, or/and ammonium salt of peroxymonosulfuric acid. More preferably, the salt of peroxymonosulfuric acid includes potassium hydrogen peroxymonosulfate.

Other sulfur-containing candidates for the active-oxygen-releasing material in the oxidizing agent for the medicinal drug of type II include thiosulfates (having S₂O₃⁻² groups). A peroxyphthalate candidate for the active-oxygen-releasing material is potassium monoperoxyphthalate. The active-oxygen-releasing material can alternatively or additionally be implemented with chlorine-oxygen-containing compounds such as hypochlorites (having ClO⁻ groups), chlorites (having ClO₂ groups), chlorates (having ClO₃⁻ groups), and perchlorates (having ClO₄⁻ groups) and with other analogous halogen compounds. One hypochlorite example is sodium hypochlorite (NaClO).

To the extent not mentioned above, the active-oxygen-releasing material in the oxidizing agent for the medicinal drug of type II may include oxygen-releasing salts such as alkali metal salts (particularly potassium, sodium, and lithium salts), alkaline earth metal salts (particularly calcium and magnesium salts), and ammonium salts of other inorganic and organic acids. Other candidates for the active-oxygen-releasing material are the alkali metal and ammonium salts of permanganic acid (HMnO₄), especially potassium permanganate (KMnO₄) but also potentially lithium permanganate (LiMnO₄), sodium permanganate (NaMnO₄), rubidium permanganate (RuMnO₄), cesium permanganate (CsMnO₄), and ammonium permanganate (NH₄MnO₄). Insofar as the drug of type II is provided in a therapeutically inactive pharmaceutically acceptable liquid carrier, again normally water, all of these candidates for the active-oxygen-releasing material need to be soluble, e.g., water soluble, in the carrier, colloidably suspendable in the carrier, or in liquid form so as to be miscible or emulsible with the carrier.

The oxidizing agent for the medicinal drug of type II may further include peroxide (O—O⁻² anion) including hydrogen peroxide (H₂O₂), oxide (O⁻² anion) including suboxide (also O⁻² anion), chromium trioxide (CrO₃), osmium tetroxide (OsO₄), and chlorine dioxide (ClO₂), dioxygenyl compound (O₂⁺ ion), oxyanion compound (AₓO_{y}^{-z} ion where A is an element and z is a charge number), peroxydisulfate (also denominated as persulfate) (S₂O₈⁻² anion) including ammonium peroxydisulfate ((NH₄)₂S₂O₈), potassium peroxydisulfate (K₂S₂O₈), and sodium peroxydisulfate (Na₂S₂O₈), sulfoxide (R—S(═O)—R′), sulfite (SO₂⁻³ anion), sulfur dioxide (SO₂), nitrate (NO₃ anion), chromate (CrO₄⁻² anion) including pyridinium chlorochromate, dichromate (Cr₂O₇⁻² anion), phosphite (PO₃⁻³ anion), hypophosphite (H₂PO₂ anion), oxoacid (or oxyacid) including sulfur oxoacid and phosphorus oxoacid, peracetic acid (CH₃CO₃H), ascorbic acid (C₆H₈O₆), formic acid (HCO₂H), or/and oxalic acid (C₂H₂O₄).

When the active-oxygen-releasing material consists at least partially of salt of peroxymonosulfuric acid such as potassium hydrogen peroxymonosulfate, the medicinal drug of type II implements the medicinal drug of type I for the situation in which the drug of type I is formed with salt of peroxymonosulfuric acid and one or more other components aside from material closely bonded to the salt of peroxymonosulfuric acid. More particularly, when the salt of peroxymonosulfuric acid consists of potassium hydrogen peroxymonosulfate provided as a component of potassium monopersulfate triple salt composition, the drug of type II implements the drug of type I for the situation in which the drug of type I is formed with potassium hydrogen peroxymonosulfate and one or more components other than the potassium hydrogen sulfate and the potassium sulfate in potassium monopersulfate triple salt composition. All of the preceding comments about the drug of type I then apply to the drug of type II. The composite drug of types I and II is referred to here as the medicinal drug of “type I/II”.

The medicinal drug of type I/II is preferably formed with potassium hydrogen peroxymonosulfate as the active oxygen-releasing material, sodium chloride as the inorganic halide, sodium hexametaphosphate as the alkali metal phosphate, sulfamic acid, malic acid as the non-reducing organic acid, and sodium dodecylbenzene sulfonate as the surfactant. The potassium hydrogen peroxymonosulfate in this formulation is normally provided as a component of potassium monopersulfate triple salt composition. The mass fractions of potassium hydrogen peroxymonosulfate, potassium hydrogen sulfate, and potassium sulfate in potassium monopersulfate triple salt composition (KHSO₅)ₓ(KHSO₄)_{y}(K₂SO₄)_{z} are approximately 45%, 25%, and 30% in this formulation so that mole fractions x, y, and z are respectively approximately 46%, 28%, and 26%.

As mentioned above, neither potassium hydrogen sulfate nor potassium sulfate readily releases oxygen under the same conditions at standard temperature and pressure for which potassium hydrogen peroxymonosulfate readily releases oxygen. Consequently, the potassium hydrogen sulfate and potassium sulfate in potassium monopersulfate triple salt composition of potassium monopersulfate triple salt composition constitute inactive material of the material used to form the preceding preferred formulation of the medicinal drug of type I/II.

The number Mₚ of moles of each component Cₚ used in forming a multi-component product consisting of a total of M_{T} moles is:

Mₚ=F_{Mp}M_{T} (13)

where F_{Mp} again is the mole fraction of component Cₚ and where the sum of all mole fractions F_{Mp} again equals 1. The molar ratio R_{Mb/a} is the ratio of the number M_{b} of moles of a component C_{b} in the product to the number Mₐ of moles of a component C_{b} in the product. Using Eq. 13, molar ratio R_{Mb/a} is:

R_{Mb/a}=F_{Mb}/F_{Ma} (14)

The parameter F_{Mb}/F_{Ma} is the mole fraction ratio R_{FMb/}a, i.e., the ratio of the mole fraction F_{Mb} of component C_{b} in the product to the mole fraction F_{Ma} of component Ca in the product. Hence, mole fraction ratio R_{FMb/a} equals molar ratio R_{Mb/a}.

Using Eq. 8, mole fraction ratio R_{FMb/a} and molar ratio R_{Mb/a} are given as:

$${R}_{FMb / a} = {R}_{Mb / a} = ( \frac{{F}_{mb}}{{F}_{ma}} ) ( \frac{{W}_{a}}{{W}_{b}} ) \qquad ( 15 )$$

where Fₘₐ and F_{mb} are the respective mass fractions of components Cₐ and C_{b} in the product and Wₐ and W_{b} are the respective molecular weights of components Cₐ and C_{b}. Consequently, the ratio R_{mb/a} of the mass fraction (or mass percentage) F_{Mb} of component C_{b} to the mass fraction (or mass percentage) Fₘₐ of component Cₐ is:

$${R}_{mb / a} = \frac{{F}_{mb}}{{F}_{ma}} = {R}_{FMb / a} ( \frac{{W}_{b}}{{W}_{a}} ) = {R}_{Mb / a} ( \frac{{W}_{b}}{{W}_{a}} ) \qquad ( 16 )$$

Eq. 16 can be employed to convert mole fraction (or molar) ratios into mass fraction ratios and thus into mass fractions.

Potassium hydrogen peroxymonosulfate as the active oxygen-releasing material, sodium chloride as the inorganic halide, sodium hexametaphosphate as the alkali metal phosphate, sulfamic acid, malic acid as the non-reducing organic acid, and sodium dodecylbenzene sulfonate as the surfactant are present at the following mass percentages of the overall material used to form the preceding preferred formulation of the medicinal drug of type I/II:
a. Potassium hydrogen peroxymonosulfate—normally 2-95%, preferably 6-90%, more preferably 10-60%, typically 20-25%;
b. Sodium chloride—normally 0.001-30%, preferably 0.005-7.5%, more preferably 0.01-5%, typically 0.5-1.5%;
c. Sodium hexametaphosphate—normally 2-60%, preferably 5-45%, more preferably 10-30%, typically 18%;
d. Sulfamic acid—normally 1-30%, preferably 1.5-15%, more preferably 3-10%, typically 5-10%;
e. Malic acid—normally 0.1-40%, preferably 0.5-30%, more preferably 1-20%, typically 5-10%; and
f. sodium dodecylbenzene sulfonate—normally 1-50%, preferably 7.5-37.5%, more preferably 10-25%, typically 15%.
   The remainder of the material used to form this formulation of the medicinal drug of type I/II largely consists of the potassium hydrogen sulfate and the potassium sulfate in the potassium monopersulfate triple salt composition that provides the potassium hydrogen peroxymonosulfate.

The medicinal drug of type I or II (including the medicinal drug of type I/II) may be provided in solid, semiliquid, or liquid form. The term “semiliquid” refers here to matter having properties between a solid and a liquid. The viscosity of semiliquid matter is sufficiently high that the semiliquid matter, when placed on a surface, flows slowly and does not rapidly adopt the shape of the underlying surface. This contrasts to liquid matter which, when placed on a surface, readily flows and rapidly adopts the shape of the underlying surface, typically within a few seconds for up to a kilogram of the liquid, and to solid matter which does not flow. As indicated below, a semiliquid form of the drug of type I or II normally has a dynamic (or absolute) viscosity of 5-5,000 Pa-s at 25° C.

Solid (dry) forms of the medicinal drug of type I or II are powders and tablets (pills) which disintegrate in the body when taken orally. When the drug of type I or II is provided in solid form, the drug itself is in solid form and is normally dispersed largely throughout a therapeutically inactive pharmaceutically acceptable solid carrier. Powder implementations of the drug of type I or II may be enclosed in water-soluble capsules (small closed containers such as jackets, sachets, films, and the like) which disintegrate in the body when taken orally. Coating agents (such as sugar, gelatin, hydroxypropyl cellulose, and hydroxypropylmethyl cellulose phthalate) may be variously provided in one or more layers or films on the tablets and capsules. The tablets and capsules can be structured to provide metered-release forms of the drug of type I or II.

The following materials can be variously admixed into solid powder and tablet forms of the medicinal drug of type I or II: vehicles (such as lactose, mannitol, glucose, microcrystalline cellulose, and starch) which help deliver the drug ingredients, binders (such as hydroxypropyl cellulose, polyvinylpyrrolidone, magnesium metasilicate aluminate, sodium stearate, polyoxyethylene and mixtures thereof), lubricants (such as magnesium stearate, polyethylene glycol, and sodium benzoate), stabilizing agents, solution adjuvants (such as glutamic acid and aspartic acid), water-absorbable materials (such as fumed silica, sodium carbonate, magnesium carbonate, and potassium carbonate) for reducing moisture, and effervescents (such as cellulose calcium glycolate, bicarbonate, carbonate, sodium bicarbonate, sodium carbonate, and citric, adipic, and tartaric acids or other similar organic acids) for releasing carbon dioxide to assist with effervescence in causing the solid drug material to disintegrate. Carbon dioxide and sodium bicarbonate may also assist in therapeutic activity. Additionally, dyes, other coloring agents, flavoring agents, fragrances, corrosion inhibitors, activity indicators, and organic activators can be applied to the solid forms of the drug of type I or II.

Semiliquid forms of the medicinal drug of type I or II include gels, creams, pastes, and ointments. When the drug of type I or II is provided in semiliquid form, the drug is normally dispersed throughout a therapeutically inactive pharmaceutically acceptable carrier, normally a semiliquid carrier. Although the carrier is normally semiliquid, the drug of type I or II can itself be solid, semiliquid, or even liquid if the drug is a suitable small mass percentage of the carrier. In a typical semiliquid form of the drug of type I or II, the drug itself consists of solid particles. The semiliquid drug of type I or II may be provided with various additives such as wetting agents, emulsifying agents, dyes, other coloring agents, flavoring agents, fragrances, corrosion inhibitors, activity indicators, organic activators, stabilizers, and buffering agents.

Liquid forms of the medicinal drug of type I or II include solutions, suspensions, lotions, emulsions, liniments, syrups, elixirs, and tinctures. In some cases, the dividing line between liquid and semiliquid forms of the drug of type I or II is unclear. When the drug of type I or II is provided in liquid form, the drug is typically dispersed throughout a therapeutically inactive pharmaceutically acceptable liquid carrier. Similar to semiliquid forms of the drug of type I or II, the drug itself can be solid, semiliquid, or liquid form. In a typical liquid form of the drug of type I or II, the drug itself consists of solid particles dissolved or colloidably suspended in the carrier. The liquid carrier is normally water (purified and/or distilled) but can be ethanol or a mixture of ethanol and water. The liquid drug of type I or II may be provided with additives such as wetting agents, suspending agents, emulsifying agents, dyes, other coloring agents, flavoring agents, fragrances, corrosion inhibitors, activity indicators, organic activators, stabilizing agents (such as sodium sulfate), and buffering agents.

As discussed further below, liquid forms of the medicinal drug of type I or II can be administered by injection or spraying. In addition to water or/and ethanol, the liquid carrier for drug injection may include one or more of vegetable oil, propylene glycol, polyethylene glycol, solution adjuvants (such as glutamic acid and aspartic acid), and soothing agents. The liquid carrier for drug spraying may include isotonic buffers (such as sodium chloride, sodium citrate, and citric acid).

The peroxymonosulfate used in forming the medicinal drug of type I can be combined with any other component of the drug and with a therapeutically inactive pharmaceutically acceptable carrier for the drug according to various techniques. The oxidizing agent, preferably peroxymonosulfate, used in forming the medicinal drug of type II can similarly be combined with the other components of the drug and with a therapeutically inactive pharmaceutically acceptable carrier for the drug according to various techniques. In the following description of such combining techniques for the medicinal drugs of types I and II, the term “peroxymonosulfate material” for the drug of type I means both the peroxymonosulfate, when it is provided as substantially a single salt, and a multiple salt composition, such as potassium monopersulfate triple salt composition for potassium hydrogen peroxymonosulfate, which contains the peroxymonosulfate as a component. The term “oxidizing agent material” for the drug of type II means both (a) the active oxygen-releasing material when the oxidizing agent is provided as substantially a single material and (b) a multi-material composition which contains the active oxygen-releasing material as a component. When the oxidizing agent consists substantially solely of peroxymonosulfate, the peroxymonosulfate material is the oxidizing agent material.

In one drug formation technique, the carrier, the peroxymonosulfate material, and any other component of the medicinal drug of type I are initially provided in solid particulate form. This technique is applicable to the medicinal drug of type II in a variation in which the carrier, the oxidizing agent material, and the other components of the drug are initially provided in solid particulate form. The particles for both the drug of type I and the drug of type II are mixed together to form a powdery substance. The mixing is normally performed so that the particles of the drug are distributed largely uniformly throughout the carrier. The drug of type I or II is then available for use as a powder. If desired, the powder can be inserted into water-soluble capsules.

If the medicinal drug of type I or II is to be in solid tablet form, the powdery substance is suitably processed to create solid tablets, including tablets that provide metered-release forms of the drug after it is administered. The processing may include providing the tablets with suitable coatings.

Largely the same procedure can be followed if the medicinal drug of type I or II is to be provided in a therapeutically inactive pharmaceutically acceptable semiliquid carrier such as a cream, gel, ointment, or foam except that the powdery substance is suitable processed to form the semiliquid carrier with the drug particles distributed throughout it. Alternatively, the carrier can initially be provided in semiliquid form with the drug of type I or II furnished in solid particulate form. If the drug of type I contains at least one component besides the peroxymonosulfate material, the particles of the drug components are mixed together. Since the drug of type II has multiple components, its components are simply mixed together. The particles of the drug of type I or II are then mixed into the carrier. The mixing for the drug of type I or II is normally performed so that the drug particles are distributed largely uniformly the semiliquid material of the carrier.

In a further drug formation technique where the carrier is to be a liquid and where the medicinal drug of type I is to be formed with at least one component besides the peroxymonosulfate material, the components of the drug are provided in solid particulate form. This technique is applicable to the medicinal drug of type II in a variation in which the oxidizing agent material and other components of the drug are initially provided in solid particulate form. The particles of the components of the drug of type I or II are mixed together to form the drug of type I or II as a powdery substance. The powdery drug is then combined with the liquid carrier in such a manner as to be distributed throughout the carrier.

The carrier is again a liquid in yet another drug formation technique. The peroxymonosulfate material for the medicinal drug of type I is provided in solid particulate form and is mixed into the liquid carrier. If the drug of type I is to be formed with at least one component besides the peroxymonosulfate material, each additional component is provided in solid particulate form or in liquid form. Each additional component for the drug of type I is then mixed into the liquid carrier separate from the peroxymonosulfate material. This technique is applicable to the medicinal drug of type II in a variation in which the oxidizing agent material is again initially provided in solid particulate form and in which each other components of the drug initially provided in solid particulate form or in liquid form. Each additional component for the drug of type II is mixed into the liquid carrier separate from the oxidizing agent material. The mixing for the drug of type I or II is performed so that the particles of the drug of type I or II are distributed throughout the carrier.

When the carrier is a liquid, the particles of the peroxymonosulfate material and any other drug component provided in particulate form for the medicinal drug of type I preferably dissolve in the carrier to form a solution and are thereby distributed substantially uniformly throughout the carrier. For the medicinal drug of type II, each drug component provided in particulate form preferably dissolves in the carrier to form a solution and thus is distributed substantially uniformly throughout the carrier.

Alternatively, the particles of the peroxymonosulfate material and any other drug component provided in particulate form for the medicinal drug of type I can be suspended in the carrier to form a colloid or emulsion. As necessary, the colloid or emulsion is suitably mixed so that the particles of the peroxymonosulfate material and any other drug component provided in particulate form are distributed substantially uniformly throughout the carrier. For the medicinal drug of type II, each drug component provided in particulate form can similarly be suspended in the carrier to form a colloid or emulsion. As necessary, the colloid or emulsion is likewise suitably mixed so that the particles of the oxidizing agent material and each other drug component provided in particulate form are distributed substantially uniformly throughout the carrier.

In some of the techniques for combining the peroxymonosulfate material and at least one other component of the medicinal drug of type I with the carrier, the peroxymonosulfate may react with another drug component, typically in the carrier. If the drug of type I is to be formed with at least two components besides the peroxymonosulfate material, two or more of these other components may react with one another, again typically in the carrier. The peroxymonosulfate or/and any other drug component besides the peroxymonosulfate material may even react with the carrier. The possibility of such reaction(s) is greater when the carrier is a liquid such as water. As a result, the chemical structure of the final constituents of the drug of type I may differ from the chemical structure of the components used to form the drug. Nonetheless, the drug of type I can reasonably be described as being “formed” with the peroxymonosulfate material and each other indicated component and thus as being “formed” at least partially with the peroxymonosulfate.

As indicated above, the oxidizing agent for the medicinal drug of type II is reactable in water, typically aqueous solution, with the halide to generate halite ions. The chemical formula for a hypohalite is XOH (or HOX) where X represents a halogen such as fluorine, chlorine, bromine or iodine. A hypohalite ion thus has the chemical formula XO⁻. When the therapeutically inactive pharmaceutically acceptable carrier is a water-containing liquid and when the oxidizing agent and the halide are dissolved in the water or are suspended in colloidal form in the water, hypohalite ions are thereby produced by reaction of the oxidizing agent with the halide. Consequently, the chemical structures of some of the components of the drug of type II in the water-containing carrier differ from the chemical structures of the corresponding components used to form the drug of type II. However, the reaction does not normally cause any significant precipitation of hypohalite material, modified oxidizing agent material, or any other material. The drug of type II can reasonably be described as being “formed” with the inorganic halide, sulfamic acid, alkali metal phosphate, and the oxidizing agent, and when used, the organic acid and the surfactant.

The water for the medicinal drug of type I and II may consist of, or include, any one or more of tap water, drinking water, purified water, hypertonic water, hypotonic water, isotonic water, oxidative reductive potential water, super-oxidized water, water for injection, Milli-Q water, saline water, heavy water (water containing a higher-than-normal proportion of deuterium in the form of deuterium protium oxide or deuterium oxide), light water (water depleted of deuterium), and tritiated water (water in which hydrogen is replaced with tritium). Purified water includes deionized water, distilled water, double distilled water, United States Pharmacopeia purified water, laboratory-grade purified water, analytical-grade purified water, or/and reagent-grade purified water. The water purification techniques for the drug of type I or II include ultra performance liquid chromatography, high performance liquid chromatography, distillation, and reverse osmosis.

The additives, including stabilizers, for the medicinal drug of type I and II may include oxidative reductive potential water, super-oxidized water, activity indicators, adjuvants, anti-adherents, anti-foaming agents, antioxidants, anti-spotting agents, aromas, binders, bioenhancers, buffering agents, carriers, catalysts, chelating agents, clarifiers, coatings, corrosion inhibitors, deflocculants, diluents, dispersants, distillers, dyes, colorants, pigments, emulsifiers, enterics, enzymes, excipients, fillers, flavors, flocculants, foaming agents, glidants, hydrotropes, lubricants, preservatives, opacifying agents, organic activators, oxygen stabilizers, preservatives, scale inhibitors, sequestrants, sorbents, solubilizers, suspending agents, sweeteners, ultraviolet stabilizers, vapor barriers, viscosity modifying agents, or/and wetting agents.

Devices for dispensing, or delivering, the medicinal drug of type I or/and II include, aerosol or/and elevated pressure containers, atomizers, dry powder inhalers, foggers, mist sprayers, nebulizers, spray and steam devices, vaporizers, metered-dose inhalers or/and dry powder inhalers. The gases or/and propellants used in these dispensing or delivering devices include nitrogen (N₂), oxygen (O₂), argon (Ar), carbon dioxide (CO₂), neon (Ne), helium (He), methane (CH₄), krypton (Kr), hydrogen (H₂), nitrous oxide (N₂O), carbon monoxide (CO), xenon (Xe), ozone (O₃), nitrogen dioxide (NO₂), iodine (I₂), ammonia (NH₃), water vapor (H₂O), or/and hydrocarbons.

The medicinal drug of types I and II is administered in various ways to people afflicted with diseases and other debilitating medical conditions caused by bacterial, eukaryotic, prion, and viral pathogens, including fungal, spore-caused, and parasitic infections, and by non-pathogenic-caused inflammation. In general, the drug of type I or II can be administered to a person by any procedure or/and route which enables the drug to reach the particular location(s) afflicted with the disease to be alleviated by the drug. Depending on various factor including the nature of the disease, the administration technique can be global, i.e., systemic, or local and thus targeted at the diseased location(s).

A topical technique is often used to administer the medicinal drug of type I or II. As used herein, topical administration generally means bringing the drug of type I or II into contact with the outer surface, typically the skin, of the human body and not by introducing the drug into the human body via one or more major openings, i.e., openings other than pores, naturally present along or/and one or more openings artificially formed along the outer surface of the human body. Topical administration can be performed by manually bringing the drug of type I or II into contact with the body, by spraying, by immersion (bath or soak), by skin or/and transdermal patch coated with the drug of type I or II and an adhesive placed on the skin to deliver the drug to the skin or/and through the skin and into the bloodstream, and by other techniques which physically bring the drug into contact with the body. The drug of type I or II can be in solid powder form, semiliquid form, or liquid form during topical administration. Topical administration of the drug of type I or II on an eye, typically with the drug in liquid form, can be performed by spraying the drug into the eye, vaporizing the drug into the eye, or/and with eye drops.

The medicinal drugs of types I and II can also be administered non-topically. Non-topical administration, the converse of topical administration, generally here means introducing the drug of type I or II into the human body via one or more major openings naturally present along or/and one or more openings artificially formed along the outer surface of the human body. Non-topical techniques for administering the drug of type I or II include oral administration, intranasal administration, intraotical administration, vaginal administration, rectal administration, urethral administration, and injection alternatively referred to as infusion or parenteral administration.

Oral administration is normally done with the medicinal drug of type I or II in solid tablet, capsule, or powder form but can be done with the drug in liquid form and sometimes with the drug in semiliquid form. The tablets, capsules, and powders may provide metered release of the drug of type I or II. For oral administration of the drug of the drug of type I or II in liquid or semiliquid form, the drug can also be placed in the mouth without having the drug go substantially through the esophagus to the stomach. For instance, a liquid form of the drug of type I or II can be administered orally by spraying the drug into the mouth or by placing the drug in the mouth and then gargling. A powder or vapor containing the drug of type I or II can be orally inhaled.

Intranasal administration in which the medicinal drug of type I or II enters at least one of the nostrils is typically done with the drug of type I or II in liquid form. Intranasal administration of the drug of type I or II can be performed by inhalation into a nostril, spraying into a nostril, vaporizing the drug into a nostril, with nose drops, or/and by other techniques which physically bring the drug into contact with the inside of a nostril.

Intraotic administration can done with the medicinal drug of type I or II in solid powder form, semiliquid form, and liquid form. In intraotic administration of the drug of type I or II, the drug typically enters the ear canal. Intraotic administration of the drug of type I or II into the ear canal can be performed by manually bringing the drug into contact with the ear canal, by spraying into the ear canal, with ear drops, vaporizing the drug into the ear canal, by immersion, or/and by other techniques which physically bring the drug into contact with the ear canal. Spraying can be done with nebulizers to achieve selected dosages. Intraotic administration of the drug of type I or II also includes introducing the drug into the middle ear or/and the inner ear and thus past the eardrum.

Vaginal and rectal administration can done with the medicinal drug of type I or II in solid powder form, semiliquid form, and liquid form. Urethral administration is typically done with the drug of type I or II in liquid form. Pessaries, suppositories, enemas and the like can be variously employed in vaginal, rectal, and urethral administration of the drug of type I or II.

Administration by injection is typically done with the medicinal drug of type I or II in liquid form. Types of injection administration of the drug of type I or II include intravenous, intramuscular, subcutaneous, intracardiac, intracavernosal, intradermal, intraosseous, intraperitoneal, and intrathecal injection.

The concentration of the medicinal drug of type I or II in its solid, semiliquid, or liquid carrier is normally 0.00001-5%, preferably 0.001-3%, more preferably, 0.1-2%, typically 0.5-1%, by mass. These ranges are particularly applicable to the medicinal drug of type I/II for which the active oxygen-releasing material in the oxidizing agent for the drug of type II consists substantially of peroxymonosulfate, preferably potassium hydrogen peroxymonosulfate, used in forming the drug of type I and for which the material used in forming the drug of type I or II includes inorganic halide, alkali metal phosphate, and sulfamic acid. The specific concentration is chosen to be therapeutically effective and non-toxic when administered in the ways described above.

The time during which the medicinal drug of type I or II is in contact with the area under treatment, especially for topical administration of the drug, varies depending upon the selected route of administration, the desired therapeutic effect, and the particular disease or other debilitating medical condition being treated with the drug. The time of contact for a liquid form of the drug of type I or II is normally 1 minute-12 hours, preferably 1 minute-6 hours, more preferably 1 minute-2 hours, typically 1-30 minutes. The time of contact for a semiliquid form of the drug of type I or II is normally 1 minute-12 hours, preferably 1 minute-6 hours, more preferably 1 minute-2 hours, typically 30 minutes-1 hour. The time of contact for a solid form of the drug of type I or II is normally 1 minute-12 hours, preferably 1 minute-6 hours, more preferably 1 minute-2 hours, typically 30 minutes-1 hour. The preceding times of contact for liquid, semiliquid, and solid forms of the drug of types I and II apply to continuous administration of the drug. The time-of-contact of the drug of type I or II can be combined with metered-release forms of the drug to obtain desired therapeutic effects.

The range of administered dosages of the medicinal drug of type I or II varies depending upon the selected route of administration, the recipient's characteristics, including age, body weight, general state of health, desired therapeutic effect, the duration of the treatment, and the disease or other debilitating medical condition being treated with the drug. The drug of type I or II can, for example, be separately administered normally 1-12 times per day, preferably 1-8 times per day, more preferably 1-6 times per day, typically 1-4 times per day, at selected unit dosages or continuously administered for selected periods at selected rates by suitable continuous administration techniques, such as intravenous injection, to achieve selected total dosage amounts. Unit dosages can be combined with metered-release dosages to obtain desired therapeutic effects.

The medicinal drug of type I or II can be administered more than 12 times daily depending on the nature of the particular disease or other debilitating medical condition being treated with the drug. The concentration of the drug of type I or II in its solid, semiliquid, or liquid carrier may exceed 5% by mass in some cases. The total administered amount and administration schedule is selected so the drug of type I or II is therapeutically effective in alleviating the symptoms of the disease or other debilitating medical condition while being non-toxic or otherwise injurious to the recipient. Additionally, the drug of type I or II may be used with many other medicines and therapies.

The time period during which a person is treated with the medicinal drug of type I or II varies depending upon the desired therapeutic effect, the particular disease or other debilitating medical condition being treated with the drug, and the person's characteristics, including age, body weight, and general state of health. The treatment period is chosen to be therapeutically effective in alleviating the symptoms of the disease being treated while avoiding toxicity difficulties with the drug of type I or II. The treatment period with the drug of type I or II is normally 1 day-12 months, preferably 1 day-6 months, more preferably 1 day-3 months, and typically 1-30 days. Nonetheless, the treatment can extend over multiple years.

The treatment plan for the medicinal drug of type I or II can be a one-time treatment, daily treatments, weekly treatments, monthly treatments, yearly treatments, or/and a long term treatment plan. Depending on the severity of the debilitating condition of the person being treated with the drug of type I or II, the treatment plan varies widely as shown by the examples presented below. Treatment with the drug of type I or II can be used as a preventative measure or/and infrequently to maintain a healthy lifestyle.

The medicinal drug of type I or II is particularly useful in treating allergic rhinitis, arthritis, bronchitis, hemorrhoids, urticaria, toothache, tinea pedis, acute viral nasopharyngitis, herpes simplex, dandruff, itching, bromhidrosis, and vaginitis. The types of arthritis include osteoarthritis and gouty arthritis. The drug of type I or II may be used to treat many other diseases and otherwise debilitating medical conditions. Other uses of the drug of type I or II include uses as an analgesic for treating pain and headaches, as an antipyretic for treating fever, as a detoxifying agent for treating hypersensitivity to various drug or/and body reactions, as a cutaneous agent for treating debilitating skin conditions, as an hematologic for treating blood diseases and other cardiac conditions, as a treatment for genetic or hereditary disorders, as a palliate for reducing conditions, as an idiopathic for treating unknown causes of a condition, and as an antidote for preventing or counteracting poisons and other such toxic conditions.

Given below are examples of using the medicinal drug of type I/II for treating allergic rhinitis, osteoarthritis, gouty arthritis, bronchitis, hemorrhoids, urticaria, toothache, tinea pedis, acute viral nasopharyngitis, herpes simplex, dandruff, itching, bromhidrosis, and vaginitis. The formulation of the drug of type I/II used in these examples was the preferred formulation, mentioned above, in which the drug was formed with potassium hydrogen peroxymonosulfate as the active oxygen-releasing material, sodium chloride as the inorganic halide, sodium hexametaphosphate as the alkali metal phosphate, sulfamic acid, malic acid as the non-reducing organic acid, and sodium dodecylbenzene sulfonate as the surfactant at approximately the typical mass percentages mentioned above. A formulation approximating this formulation is commercially available in the product Virkon. This formulation of the drug of type I/II is referred to below as the “Medicine”.

The Medicine for each person infected with allergic rhinitis, osteoarthritis, gouty arthritis, bronchitis, hemorrhoids, urticaria, toothache, tinea pedis, acute viral nasopharyngitis, herpes simplex, dandruff, itching, bromhidrosis, and vaginitis is provided for topical, intranasal, oral, or/and injection administration 1-12 times per day, preferably 1-8 times per day, more preferably 1-6 times per day, typically 1-4 times per day, in liquid form at a dosage of 0.00001-5%, preferably 0.001-3%, more preferably 0.1-2%, typically 0.5-1%, for a time of contact of 1 minute-12 hours, preferably 1 minute-6 hours, more preferably 1 minute-2 hours, typically 1-30 minutes. The treatment period in each in each of the following examples is normally 1 12 months, preferably 1-6 months, more preferably 1-3 months, typically 1-30 days.

### Example A

### Allergic Rhinitis

A teenage female had allergic rhinitis. The female stated that she experienced annual episodes of itching, burning, congestion, and watering of mucosal membranes apparently resulting from hypersensitivity to plant allergens. The Medicine was provided to the female as a 1% solution for intranasal administration by spraying into the nostrils 3 times a day at 4-hour intervals for 2 weeks. The female reported that her allergic rhinitis symptoms were relieved after 3 days and that the symptoms virtually disappeared after 10 days. The female then stopped using the Medicine. The female reported that the allergy symptoms returned after 14 more days, that she resumed taking the Medicine, and that the symptoms disappeared within several more days. As a preventative measure, the female reports that she now uses the Medicine before hay fever season.

### Example B

### Osteoarthritis

A retired adult male had osteoarthritis for 10 years. The male stated that he was in constant pain throughout the day, thereby limiting his life substantially to his surroundings. The male reported that he had tried many treatments, such as NSAIDs, glucosamine, and glucocorticoids, for his osteoarthritis without effective relief. The Medicine was provided to the male as a 1% solution for topical administration via complete-body immersion (bath) for 15 minutes, with morning and night applications, for a minimum of 2 weeks. The male reported that, after 7 days of treatment, the pain was relieved greatly and that, after 14 days of treatment, the pain was very minimal. The male reports that he now uses the Medicine at least twice a week to reduce pain of osteoarthritis.

### Example C

### Gouty Arthritis

A retired adult male had gouty arthritis for 10 years. The male reported that he was in excruciating pain on random days and that previously attempted treatments for the pain were unsuccessful. The Medicine was provided to the male as a 1% solution for topical administration via complete-body immersion once a day, before bedtime, for a minimum of 20 minutes for 7 days. The male reported that the pain was greatly relieved on day 4. The male reports that he now sleeps better than in many previous years and that he continues to use the Medicine at least twice a week as a treatment and preventative.

### Example D

### Bronchitis

An adult female had severe coughing and sputum due to bronchitis. The female reported that the bronchitis had been intermittent for 10 years and that she had tried many treatments for her bronchitis and consumed substantial amounts of water daily with no success. The Medicine was provided to the female as a 0.1% solution for oral administration by spraying into the mouth 3 times per day at 4-hour intervals for 7 days. After the 7 days, the female reported that her bronchitis was greatly alleviated, that the urge to cough had diminished greatly, and that the sputum was virtually gone. The female reports that she now feels better than she has in the past 10 years.

### Example E

### Hemorrhoids

An adult female had hemorrhoids for over 5 years. The female stated that the hemorrhoids were so severe as to prevent her from working, that she was confined to bed during most of each day due to severe pain in walking and standing, and that the pain was almost unbearable during bowel movements. The Medicine was provided to the female as a 1% solution for rectal/topical administration 1-2 times a day for 14 days. For the rectal/topical administration, the female was instructed to perform bowel movements in a container filled with the Medicine to a level so that her buttocks were completely immersed in the Medicine, so that the Medicine contacted the outer layer of the anus, and so that the Medicine moved through the anus into the rectum. After each bowel movement, the Medicine in the container was to be discarded. The female reported that her hemorrhoids were greatly alleviated and that she was now able to walk far distances which she had not been able to do for over 5 years.

### Example F

### Urticaria

An adult female had a severe outbreak of urticaria apparently due to consumption of raw seafood. The female stated that she had itchy and bumpy red spots on her face, back and thighs. The female stated that she had tried many treatments for her urticaria. The Medicine was provided to the female as a 0.5% solution for topical administration via complete-body immersion for 20 minutes once a day for a few days. The female reported that the itchiness was relieved on the night of initial treatment and that her urticaria was virtually gone on day 3. The female also reported that no previous treatment for her urticaria was as fast acting as the Medicine.

### Example G

### Toothache

An adult female had a tooth extracted and was in extreme pain due to the tooth extraction. The Medicine was provided to the female as a 1% solution for oral administration by gargling for 1 minute. The female reported that the pain diminished greatly in about 30 minutes. The female further reported that she continued using the Medicine before bedtime that night and the next day during the morning and evening and that, after 2 days of treatment, the toothache pain disappeared.

### Example H

### Tinea Pedis

An adult male had tinea pedis with itching, cracking, and burning lesions of his toes. The Medicine was provided to the male as a 1% solution for topical administration via foot immersion for 10 minutes a day for several days. The male reported that his toes were healed after 5 days and that the Medicine was one of the most effective medications that he had ever used.

### Example I

### Acute Viral Nasopharyngitis

An adult female apparently had viral nasopharyngitis. She reported the typical viral nasopharyngitis symptoms of sneezing, congestion, and fatigue. The Medicine was provided to the female as a 0.1% solution (i) for intranasal administration by spraying and (ii) for oral administration by spraying, each type of administration to be performed 3 times a day at 4-hour intervals for 4 days. The female reported that the symptoms of sneezing, congestion, and restlessness had significantly decreased by day 4 and that they had virtually disappeared on the morning of day 5.

### Example J

### Herpes Simplex

An adult female had herpes simplex in the form of herpes labialis as indicated by cold sores around her mouth. The female reported that she contracted the cold sores about 3 times a year, that she felt some pain and itching from the cold sores, and that the external visibility of the cold sores made her reluctant to meet people. The female further reported the she had tried analgesics and herbal remedies to treat the cold sores without complete success and that she normally ended up waiting about 7 days for the cold sores to scab over. The Medicine was provided to the female as a 1% solution for topical administration around the mouth at 3-hour intervals 4 times a day for 7 days. The female reported that, on day 2, the cold sores had become completely flat with the pain and itchiness substantially gone and that the cold sores were barely visible on day 6.

### Example K

### Dandruff

An adult female had dandruff for about 20 years. The female stated that she avoided wearing black clothes due to the dandruff flaking and that the dandruff caused her to have low self esteem. The female reported that she had tried numerous treatments, such as zinc pyrithione, ketoconazole, selenium sulfide, and many types of herbs, to eliminate her dandruff and that (some of) these treatments did cause the amount of dandruff to decrease but that dandruff was still visible on her scalp. The Medicine was provided to the female as a 1% solution for topical administration by scrubbing the Medicine into her scalp and hair for 5 minutes and then washing her scalp and hair. The female reported that she administered the Medicine to herself in the foregoing manner 4 times a week for 2 weeks. After the 2 weeks, the female reported that the dandruff disappeared. The female reports that the dandruff has not come back.

### Example L

### Itching

An adult male had extreme itching for 5 years. The male reported that he had tried many treatments for the itching without success. The Medicine was provided to the male as a 1% solution for topical administration via complete-body immersion for 20 minutes 2 times a day for 2 weeks. The male reported that the itching had diminished by at least 50% on day 3 and that the itching was substantially gone at the end of the 2 weeks. The male reports that he now uses the Medicine twice a week as an itch preventative.

### Example M

### Bromhidrosis

An adult male had bromhidrosis in the form of strong foot odor. The male reported that he tried treatments such as cologne and deodorant for the bromhidrosis but that these treatments caused his skin to swell and redden. The Medicine was provided to the male as a 1% solution for topical administration via foot bath immersion for 10 minutes in the morning and evening for 7 days. After 5 days, the male reported that his foot odor had disappeared. The male further reported that the foot odor returned about 30 days later, that he then used the Medicine twice in 2 days, and that the foot odor again went away.

### Example N

### Vaginitis

An adult female had vaginitis for 3 years. The female reported vaginitis symptoms of inflammation, burning, itching, and swelling after intercourse. The female reported that she has tried many treatments, such as topical antibiotics, anti-fungal creams, and hydrocortisone, for the vaginitis but without significant success. The Medicine was provided to the female as a 1% solution for vaginal/topical administration via vaginal immersion for 10 minutes in the morning and evening for 2 weeks. The female reported that the burning and itching in the vaginal area were significantly reduced on day 2 and that the burning, itching and inflammation in the vaginal area were greatly alleviated on day 10. The female further reports that she continues to use the Medicine after intercourse.

A metered-release product in accordance with the invention is a disintegrable composition formed with solid material and suitable for introduction into a liquid, typically water. The disintegrable composition consists at least partially of active material formed with the medicinal drug of type I or II. Immediately prior to being introduced into the liquid, the disintegrable composition has a mass m_{DC} of an initial mass value m_{DCI}. The disintegrable composition is of such a structure that the composition gradually disintegrates upon introduction into the liquid for enabling particles of the composition to disperse into the liquid. Mass m_{DC} of the disintegrable composition is 10% to 90% of initial mass value m_{DCI} 1 hour to 100 days after the composition is introduced into the liquid. The rate at which the composition disintegrates is adjusted to accommodate various situations.

Relatively fast metered release, e.g., over a small number of hours to a small number of days, of the medicinal drug of type I or II is appropriate for some situations such as that in which the disintegrable composition containing the drug of type I or II is provided in sufficiently large solid form, such as a tablet or a capsule containing a dry powder of the disintegrable composition, for ingestion by a person. For such fast metered release, mass m_{DC} of the disintegrable composition is 10% to 90% of initial mass value m_{DCI} 1 hour to 24 hours after the composition is introduced into the liquid. Preferably, mass m_{DC} of the disintegrable composition for fast metered release is 10% to 90% of initial mass value m_{DCI} 2 hours to 12 hours after the composition is introduced into the liquid. In regard to a capsule containing dry powder of the disintegrable composition, the preceding times begin at the point that the capsule has disintegrated sufficiently so as to expose the active material to the liquid.

Relatively slow metered release, e.g., over multiple months to a small number of years, of the medicinal drug of type I or II is appropriate for other situations such as that in which the potency of a liquid form of the drug of type I or II is to be maintained suitably high for multiple months to a year or more. For such slow metered release, mass m_{DC} of the disintegrable composition is 10% to 90% of initial mass value m_{DCI} 25 days to 100 days after the composition is introduced into the liquid. Preferably, mass m_{DC} of the disintegrable composition for slow metered release is 10% to 90% of initial mass value m_{DCI} 45 days to 100 days after the composition is introduced into the liquid. For slow metered release, the disintegrable composition is generally provided as one or more solid pieces, such as tablets, of significant size. The liquid is normally water, preferably high-purity water, more preferably deionized water, in one embodiment of slow metered release of the drug of type I or II.

In another embodiment of slow metered release of the medicinal drug of type I or II, the liquid is an initial liquid composition prepared by dispersing a selected amount of the drug of type I or II throughout a therapeutically inactive carrier, likewise normally water, preferably high-purity water, more preferably deionized water. The initial liquid composition thereby has a suitably high initial potency. The subsequent introduction of the disintegrable composition into the initial liquid composition converts it into a further liquid composition containing the drug of type I or II. The disintegration characteristics of the disintegrable composition are chosen so that it disintegrates in such a way in the further liquid composition as to provide it with sufficient active-oxygen-containing matter of the drug of type I or II to generally compensate for active oxygen lost from the further composition as time passes. This enables the potency of the further liquid composition to be maintained suitably high for an extended period so that the further composition has extended shelf life.

The ionic purity of a liquid is generally gauged by its conductivity or its resistivity, the inverse of conductivity. The amount of ionization in the liquid increases as its conductivity increases or its resistivity decreases, and vice versa. The conductivity of the deionized water (a) into which the disintegrable composition is introduced in the first embodiment of slow metered release of the medicinal drug of type I or II and (b) into which the selected amount of the drug of type I or II is introduced in the second embodiment of slow metered release of the drug of type I or II to form the initial liquid composition is normally no more than 10 μS/cm, preferably no more than 1 μS/cm, more preferably no more than 0.1 μS/cm, at 25° C. Equivalently, the resistivity of the deionized water at 25° C. is at least 0.2 MΩ-cm, preferably at least 1 MΩ-cm, more preferably at least 10 MΩ-cm, (a) prior to introducing the disintegrable composition into the deionized water in the first embodiment of slow metered release and (b) prior to introducing the selected amount of the drug of type I or II into the deionized water in the second embodiment of slow metered release. The carrier for the initial liquid composition can include oxidative reductive potential water or/and super-oxidized water.

Metered release of the medicinal drug of type I or II at a release rate intermediate to fast and slow metered releases is appropriate for yet other situations. For such intermediate-rate metered release, mass m_{DC} of the disintegrable composition is 10% to 90% of initial mass value m_{DCI} 1 days to 24 days after the composition is introduced into the liquid. Preferably, mass m_{DC} of the disintegrable composition for intermediate-rate metered release is 10% to 90% of initial mass value m_{DCI} 5 days to 20 days after the composition is introduced into the liquid. The liquid for intermediate-rate metered release is normally water, preferably high-purity water, more preferably deionized water, and can include oxidative reductive potential water or/and super-oxidized water.

The disintegrable composition can, as indicated above, be provided in the form of solid pieces, e.g., tablets, of significant size, or as a dry powder. The average diameter of such solid pieces of significant size is normally at least 10 mm, preferably 15 mm, more preferably 20 mm. The average diameter of the particles of the powder is normally no more than 400 μm, preferably no more than 300 μm, more preferably no more than 200 μm. Selected amounts of the powder of the disintegrable composition are commonly respectively encased with coatings to form capsules suitable for introduction into the liquid. The coatings normally disintegrate rapidly after the capsules are introduced into the liquid. In particular, the coatings typically disintegrate in several minutes and thus much faster than the disintegrable composition. The coatings are normally substantially chemically non-reactive with the active material when it is dry or in water. By providing the disintegrable composition in the form of solid material, the composition has an extended shelf life.

The disintegrable composition is fabricated so as to disintegrate in the manner described above. In this regard, the disintegrable composition normally includes solid support material which provides, or assists in providing, the composition with disintegration characteristics that enable the composition to disintegrate in the above-described manner. Consequently, the support material is appropriately combined with the active material containing the medicinal drug of type I or II. The support material serves as a solid carrier for the active material.

The disintegration characteristics can also be controlled by providing pieces containing the disintegrable composition, whether in tablet form, capsule form, or some other form, with coatings which partially encase the composition-containing pieces and which do not disintegrate significantly in the liquid. For instance, pieces containing the disintegrable composition can be readily furnished with partial non-disintegrable coatings that enable the composition to disintegrate at a relatively constant rate. In addition, the disintegration characteristics can be controlled by providing pieces containing the disintegrable composition with coatings, partial or full, which disintegrate at certain places but not others when the composition-containing pieces are placed in the liquid.

For the situation in which the active material in the solid disintegrable composition is implemented with the medicinal drug of type I, the active material contains salt of peroxymonosulfuric acid, preferably potassium hydrogen peroxymonosulfate. The support material is normally substantially chemically non-reactive with the salt of peroxymonosulfuric acid and with reaction product of the salt of peroxymonosulfuric acid and any other material in this implementation of the disintegrable composition when it is dry or combined with water. The active material in the implementation of the disintegrable composition using the drug of type I may include one or more of inorganic halide, metal phosphate, sulfamic acid, a non-reducing organic acid, and a surfactant as additional materials. The support material is then normally substantially chemically non-reactive with each additional material and with reaction product of each additional material and other material in this implementation of the disintegrable composition when it is dry or combined with water.

When the active material in the disintegrable composition is implemented with the medicinal drug of type II, the active material contains inorganic halide an oxidizing agent reactable in water with the inorganic halide to generate hypohalite ions. The support material is normally substantially chemically non-reactive with the oxidizing agent and with reaction product of the oxidizing agent and other material in this second implementation of the disintegrable composition when it is dry or combined with water. The active material in the implementation of the disintegrable composition using the drug of type II may include one or more of metal phosphate, sulfamic acid, a non-reducing organic acid, and a surfactant as additional materials. The support material is then similarly normally substantially chemically non-reactive with each additional material and with reaction product of each additional material and other material in this second implementation of the disintegrable composition when it is dry or combined with water.

All of the above-mentioned additional materials, except possibly the surfactant, are normally present in each implementation of the disintegrable composition. Candidates for the inorganic halide, the metal phosphate, the non-reducing acid, and the surfactant in either implementation of the disintegrable composition and for the oxidizing agent in the implementation using the drug of type II are presented above.

Preparation of the disintegrable composition entails providing the active material and any other component of the composition in a structure that enables the composition, upon being later introduced into the liquid, to gradually disintegrate in the general metered-release manner specified above. More particularly, the support material (when present) and the components of the active material are so combined to achieve the specified disintegration characteristics.

The disintegrable composition is administered to a person to treat a debilitating medical condition of the person or/and to prevent the person from contracting the debilitating medical condition in various ways depending on the nature of the medical condition. In one administration technique, the person simply ingests one or more pieces, e.g., one or more tablets or capsules, containing the disintegrable composition. Liquid, normally water, in the person's body then serves as the liquid in which each piece containing the disintegrable composition disintegrates to form a further composition containing the medicinal drug of type I or II and thus the active material.

In another administration technique, one or more solid pieces, e.g., again one or more tablets or capsules, containing the disintegrable composition are introduced into a liquid, again normally water, that serves as a carrier for the disintegrable composition. Each solid piece containing the disintegrable composition disintegrates in the liquid carrier to form a further composition, normally liquid, containing the medicinal drug of type I or II and thus again the active material. Material of the further composition is variously administered topically, orally, intranasally, intraotically, vaginally, rectally, urethrally, and by injection as further described above. To the extent that the solid pieces containing the disintegrable composition have not disintegrated, the remaining portions of the composition-containing pieces are normally not administered to a person. Depending on how fast the disintegration of the disintegrable composition proceeds, the disintegration of the composition may take a considerable period of time, e.g., hours to days to months, after the first administration of the further composition containing the drug of type I or II. The potency of the further composition thereby stays at a suitably high level for an extended time period.

The debilitating medical condition in any of the administration techniques using the disintegrable composition can be any of the above-identified debilitating medical conditions, namely bacterial, eukaryotic, prion-caused, and viral infections, including fungal, spore-caused, and parasitic infections, and non-pathogenic-caused inflammation and specifically including allergic rhinitis, arthritis, bronchitis, hemorrhoids, urticaria, toothache, tinea pedis, acute viral nasopharyngitis, herpes simplex, dandruff, itching, bromhidrosis, and vaginitis.

A semiliquid product in accordance with the invention is a semiliquid composition consisting at least of a carrier and active material dispersed largely throughout the carrier. The active material is implemented with the medicinal drug of type I or II. The semiliquid composition normally has a dynamic viscosity μ of at least 5 Pa-s at 25° C. Dynamic viscosity μ of the semiliquid composition is preferably at least 10 Pa-s, more preferably at least 20 Pa-s, even more preferably at least 50 Pa-s, at 25° C. Additionally, dynamic viscosity μ of the semiliquid composition is normally no more than 5,000 Pa-s, preferably no more than 2,000 Pa-s, more preferably no more than 500 Pa-s, even more preferably no more than 150 Pa-s, at 25° C.

The carrier for the semiliquid composition is typically semiliquid. However, the carrier can be liquid if the amount of active material present in the carrier is sufficiently high to cause the composition to be semiliquid, i.e., to cause the composition to have the dynamic viscosity characteristics specified above. The carrier often includes water, preferably high-purity water, more preferably deionized water. The carrier can include oxidative reductive potential water or/and super-oxidized water.

The semiliquid composition is prepared by combining the active material and the carrier so that the active material is dispersed largely throughout the carrier and so that the composition is semiliquid. For instance, the active material can be introduced into the carrier. Alternatively or/and additionally, the carrier can be poured on or otherwise placed on the active material. In either case, suitable mixing of the active material and the carrier is typically performed to enable the active material to be dispersed largely throughout the carrier.

The medicinal drug of type I or II may undergo chemical reactions in the course of combining the active material and the carrier. When the active material in the semiliquid composition is implemented with the drug of type I, the active material contains salt of peroxymonosulfuric acid or/and reaction product of the salt of peroxymonosulfuric acid and other material of the composition. The salt of peroxymonosulfuric acid preferably is potassium hydrogen peroxymonosulfate. The active material in the implementation using the drug of type I may include one or more of the following additional materials: (a) inorganic halide or/and reaction product of the inorganic halide and other material of the composition, (b) metal phosphate or/and reaction product of the metal phosphate and other material of the composition, (c) sulfamic acid or/and reaction product of sulfamic acid and other material of the composition, (d) a non-reducing organic acid or/and reaction product of the organic acid and other material of the composition, and (e) a surfactant or/and reaction product of the surfactant and other material of the composition.

When the active material in the semiliquid composition is implemented with the medicinal drug of type II, the active material contains (a) inorganic halide or/and reaction product of the inorganic halide and other material of the semiliquid composition and (b) the oxidizing agent or/and reaction product of the oxidizing agent and other material of the composition where the oxidizing agent is reactable in water with the inorganic halide to generate hypohalite ions. The active material in the implementation using the drug of type II may include one or more of the following additional materials: (a) metal phosphate or/and reaction product of the metal phosphate and other material of the composition, (b) sulfamic acid or/and reaction product of sulfamic acid and other material of the composition, (c) a non-reducing organic acid or/and reaction product of the organic acid and other material of the composition, and (d) a surfactant or/and reaction product of the surfactant and other material of the composition.

The above-mentioned reaction products are variously present in the semiliquid composition implemented with the medicinal drug of type I or II as a result of combining the drug of type I or II with the carrier. All of these additional materials, except possibly the surfactant, are normally present in each implementation of the semiliquid composition. Candidates for the inorganic halide, the metal phosphate, the non-reducing acid, and the surfactant in either implementation of the semiliquid composition and for the oxidizing agent in the implementation using the drug of type II are presented above.

It is expected that the semiliquid composition will undergo fewer chemical reactions and other physical/chemical phenomena which cause the potency of the medicinal drug of type I or II in the composition to decrease with time. Consequently, the shelf life of the semiliquid composition is expected to be extended. At the same time, the semiliquid nature of the composition, especially when viscosity μ of the composition in the range of 5-5,000 Pa-s at 25° C., enables the composition to be readily administered to humans.

Material of the semiliquid liquid composition is administered to a person sufficiently to treat a debilitating medical condition of the person or/and to prevent the person from contracting the debilitating medical condition. The administration of material of the semiliquid composition is variously performed topically, orally, intranasally, intraotically, vaginally, rectally, urethrally, and by injection as further described above. The debilitating medical condition can be any of the above-identified debilitating medical conditions, namely bacterial, eukaryotic, prion-caused, and viral infections, including fungal, spore-caused, and parasitic infections, and non-pathogenic-caused inflammation and specifically including allergic rhinitis, arthritis, bronchitis, hemorrhoids, urticaria, toothache, tinea pedis, acute viral nasopharyngitis, herpes simplex, dandruff, itching, bromhidrosis, and vaginitis.

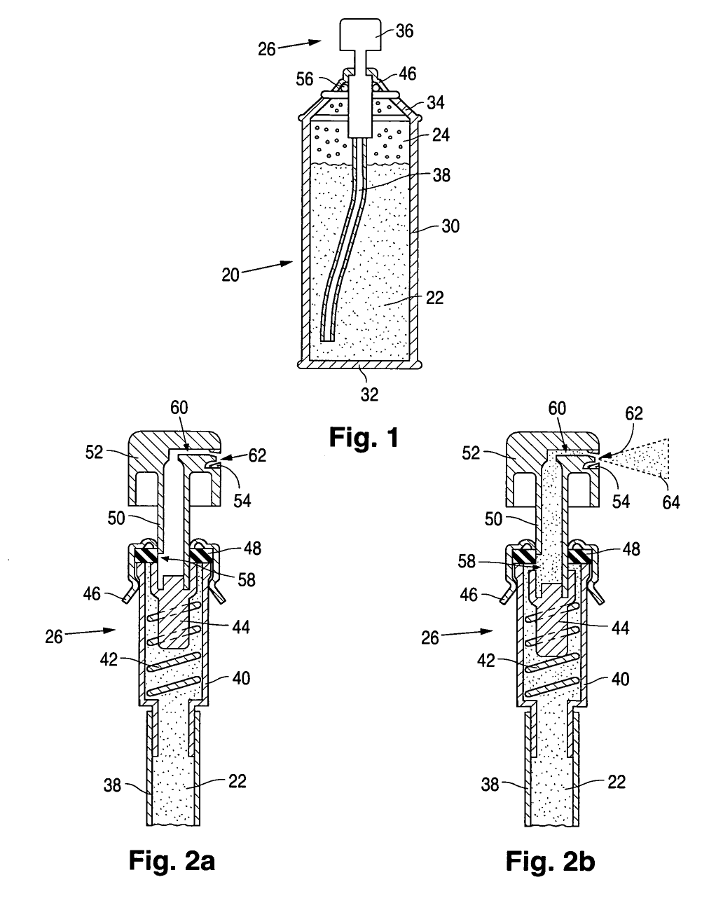

FIG. 1 illustrates a product in accordance with the invention for dispensing, or delivering, the medicinal drug of type I or II provided in a therapeutically inactive liquid carrier for situations in which the liquid form of the drug of type I or II needs extended shelf life. In describing the product of FIG. 1, all references to the drug of type I or II include any of the variations of the liquid form of drug of type I or II described below.

The extended-shelf-life product in FIG. 1 is a container assembly consisting of a container 20, a liquid composition 22, a pressurant gas 24, and a dispenser 26 hermetically attached to the top of container 20. Liquid composition 22 and pressurant gas 24 are situated inside container 20. Pressurant gas 24, located above liquid composition 22, exerts an average internal pressure of more than 1 atm (760 torr), typically 2-8 atm, on composition 22. Dispenser 26 controllably dispenses material of liquid composition 22 from container 20.

Liquid composition 22 includes the therapeutically inactive liquid carrier and active material dispersed largely throughout the carrier. The active material consists of the medicinal drug of type I or II. Pressurant gas 24 consists of a gas which is substantially non-reactive with the drug of type I or II and with its carrier. In one embodiment of the container assembly of FIG. 1, liquid composition 22 consists substantially solely of the carrier and the active material formed with the drug of type I or II. In another embodiment of the container assembly of FIG. 1, a liquid form of some of the gas which forms pressurant gas 24 is also present in liquid composition 22.

The liquid carrier for the medicinal drug of type I or II in liquid composition 22 is usually water. Certain materials in normal water, such as tap water, can cause the water-carrier form of the drug of type I or II to undergo reactions which cause the drug's potency to decrease with time. In the container assembly of FIG. 1, the loss of potency with time is reduced by implementing the water carrier for the drug of type I or II with high-purity water, preferably deionized water. Prior to combining the deionized water and the solid form of the drug of type I or II to create the liquid form of the drug of type I or II for the container assembly of FIG. 1, the deionized water has the conductivity and resistivity characteristics mentioned above. The liquid carrier can include oxidative reductive potential water or/and super-oxidized water.

Ions of material of the medicinal drug of type I or II are inevitably produced when the deionized water and the solid form of the drug of type I or II are combined to create the drug's liquid form. The amount of ionization, per unit volume, in the liquid form of the drug of type I or II is therefore inevitably greater than the amount of ionization, per unit volume, in the deionized water. The conductivity of the liquid form of the drug of type I or II can be characterized as consisting of (a) a drug-related part arising from ions of material of the drug of type I or II and (b) a water-related part arising from other ions in the water carrier. The water-related part of the conductivity of the liquid form of the drug of type I or II for the container assembly of FIG. 1 is essentially the conductivity of the deionized water prior to combining it with the solid form of the drug of type I or II.

For the situation in which the active material in liquid composition 22 is implemented with the medicinal drug of type I, the active material contains salt of peroxymonosulfuric acid or/and reaction product of the salt of peroxymonosulfuric acid and other material of composition 22. The salt of peroxymonosulfuric acid preferably is potassium hydrogen peroxymonosulfate. The active material in the implementation of liquid composition 22 using the drug of type I may include one or more of the following additional materials: (a) inorganic halide or/and reaction product of the inorganic halide and other material of composition 22, (b) metal phosphate or/and reaction product of the metal phosphate and other material of composition 22, (c) sulfamic acid or/and reaction product of sulfamic acid and other material of composition 22, (d) a non-reducing organic acid or/and reaction product of the organic acid and other material of composition 22, and (e) a surfactant or/and reaction product of the surfactant and other material of composition 22.

When the active material in liquid composition 22 is implemented with the medicinal drug of type II, the active material contains (a) inorganic halide or/and reaction product of the inorganic halide and other material of composition 22 and (b) the oxidizing agent or/and reaction product of the oxidizing agent and other material of composition 22. The oxidizing agent is reactable in water with the inorganic halide to generate hypohalite ions. The active material in the implementation of liquid composition 22 using the drug of type II may include one or more of the following additional materials: (a) metal phosphate or/and reaction product of the metal phosphate and other material of composition 22, (b) sulfamic acid or/and reaction product of sulfamic acid and other material of composition 22, (c) a non-reducing organic acid or/and reaction product of the organic acid and other material of composition 22, and (d) a surfactant or/and reaction product of the surfactant and other material of composition 22.

The above-mentioned reaction products are variously present in liquid composition 22 implemented with the medicinal drug of type I or II as a result of combining the drug of type I or II with the liquid carrier. All of the above-mentioned additional materials, except possibly the surfactant, are normally present in each implementation of liquid composition 22. Candidates for the inorganic halide, the metal phosphate, the non-reducing acid, and the surfactant in either implementation of liquid composition 22 and for the oxidizing agent in the implementation using the drug of type II are presented above.

Visible light and certain other types of radiation, such as UV radiation, that impinge on the liquid form of the medicinal drug of type I or II, can also cause the drug's potency to decrease with time. The potency loss due to incident visible light and/or incident UV radiation is reduced by designing container 20 to substantially block the transmission of visible light and UV radiation incident on container 20 from outside container 20. The radiation-transmission blocking can occur by reflection of the incident visible-light and UV radiation at the outside surface of container 20 and/or absorption of the incident radiation by the material of container 20. As a result, visible light and UV radiation incident on the outside surface of container 20 does not significantly reach the liquid form of the drug of type I or II inside container 20 so as to cause the drug's potency to decrease with time.

Container 20 is designed to withstand the average internal pressure of more than 1 atm exerted by pressurant gas 24. The inside of container is substantially non-reactive with liquid composition 22 and thus substantially non-reactive with the medicinal drug of type I or II and its carrier. The inside of container is also substantially non-reactive with pressurant gas 24. To achieve these capabilities and to achieve the desired light-blocking capability, container 20 normally consists primarily of suitably strong metal, such as steel, aluminum, or/and tin, or/and suitably strong electrical insulating material such as silica (silicon dioxide) or plastic. If the metal or/and insulating material should react significantly with liquid composition 22 or pressurant gas 24, the metal or/and insulating material can be coated along the inside of container 20 with material which is substantially non-reactive with composition 22 and pressurant gas 24.

Visible light and UV radiation incident on the outside of container 20 is normally substantially reflected along most of the container's outside surface. If the metal or/and insulating material that normally primarily forms container 20 is not sufficiently light reflective, the metal or/and insulating material can be coated along the outside of container 20 with material which is sufficiently light reflective. The light-reflective material can be coated with light-transparent material so that visible light and UV radiation incident on the outside of container 20 largely pass through the transparent coating, are reflected by the underlying light-reflective material, and then pass back through the transparent coating.

Most of container 20 typically appears white or light colored as seen from outside container 20. In particular, most of the outside of container 20 appears white or light colored except for dark-colored areas that provide printed information about the medicinal product of FIG. 1. This coloring can, for instance, be achieved by providing container 20 with white or light-colored information-containing labels that cover most of the container's outside surface. Advantage is thereby taken of the phenomenon that objects of light color, especially white, are generally more effective in reflecting light than are objects of dark color.

Container 20 consists of a solid side wall 30, a solid bottom wall 32 hermetically attached to the bottom of side wall 30, and a solid top wall 34 likewise hermetically attached to side wall 30. Side wall 30 is normally of cylindrical shape, typically circularly cylindrical shape, but can have other shapes. Bottom wall 32 is typically largely flat. Top wall 34 typically bulges upward but can be largely flat. The inside surfaces of walls 30, 32, and 34 contact liquid composition 22 and pressurant gas 24. The thicknesses of walls 30, 32, and 34 are chosen to be sufficiently great to absorb visible light and UV radiation incident on any dark areas along the outside of container 20 and to safely withstand the internal pressure of more than 1 atm, again typically 2-8 atm, exerted by pressurant gas 24.

Dispenser 26 consists of a valve 36 and a dip tube 38 which extends into liquid composition 22. Valve 36, only generally shown in FIG. 1, is illustrated in more detail in FIGS. 2a and 2b respectively in non-actuated and actuated conditions. The components of valve 36 consist of a valve housing 40, a valve spring 42, a spring cup 44, a valve cup 46, a sealing gasket 48, a valve stem 50, an actuator 52, and an orifice insert 54 configured as shown in FIGS. 2a and 2b. The hermetic sealing of dispenser 26 to container 20 is achieved with a sealing element 56 situated between valve cup 46 and top wall 34.

Valve housing 40 is connected to dip tube 38 in which part of liquid composition 22 is present. The more-than-1-atm pressure exerted by pressurant gas 24 on liquid composition 22 causes material of composition 22 to pass through dip tube 38 and enter valve housing 40. When dispenser 26 is in the non-actuated condition depicted in FIG. 2a, the top edge of valve cup 44 fully contacts the bottom of sealing gasket 48. This prevents any of liquid composition 22 from then passing through valve housing 40 and entering valve stem 50.

Valve stem 50 has an open area 58 at the bottom of stem 50. Dispenser 26 is actuated by pressing on actuator 52 so that it moves downward. The actuation of dispenser 26 as depicted in FIG. 2b causes valve cup 44 to move downward and separate from sealing gasket 48. Some of liquid composition 22 is then forced upward into valve stem 50 under the driving force provided by the more-than-1-atm pressure of pressurant gas 24. This part of liquid composition 22 enters a channel 60 in actuator 52 and passes through an orifice 62 in orifice insert 54 to form a spray 64 of composition 22.

Actuator 52 and orifice insert 54 can be alternatively configured to enable a stream of liquid composition 22 to be provided form orifice insert. Regardless of whether the container assembly of FIG. 1 dispenses liquid composition 22 in spray-form or stream form, the combination of the configuration of dispenser 26 and the dispensing of composition 22 under the driving force of the more-than-1-atm pressure of pressurant gas 24 enables dispenser 26 to substantially prevent air and other material outside container 20 from entering container 20 through dispenser 26. As a result, the potency of the liquid form of the medicinal drug of type I or II does not significantly decrease with time due to reactions with material outside container 22.

The container assembly of FIG. 1 is manufactured in generally the following manner. Container 20, dispenser 26, and sealing element 56 are separately fabricated. Liquid composition 22 is prepared by suitably combining the solid form of the medicinal drug of type I or II and its liquid carrier. If liquid composition 22 is to include a liquid form of some of the gas that forms pressurant gas 24, the liquid form of that gas is also suitably combined with the medicinal drug of type I or II and its liquid carrier. When the liquid carrier for the drug of type I or II consists of deionized water, liquid composition 22 is prepared by combining the solid form of the drug of type I or II, its liquid carrier, and, if used in composition 22, the liquid form of some of the gas that forms pressurant gas 24 in a vacuum chamber at a chamber pressure significantly below atmospheric pressure, nominally 1 atm, in order to avoid introducing air and materials in air into composition 22. The chamber pressure is normally no more than 100 torr, preferably no more than 10 torr, more preferably no more than 1 torr.

Container 20, liquid composition 22, dispenser 26, and sealing element 56 are now assembled in such a manner that composition 22 and pressurant gas 24 are introduced into container 20 and in such a manner that dispenser 26 is hermetically attached to container 20 via sealing element 56. The assembly can be done in various ways depending on various factors, including the purity characteristics of liquid composition 22.

If liquid composition 22 needs to be maintained highly pure, e.g., because the liquid carrier for the medicinal drug of type I or II consists of deionized water, composition 22 and pressurant gas 24 are introduced into container 20 in such a way as to substantially prevent composition 22 from being contaminated with air and materials in air. Dispenser 26 is subsequently hermetically attached to container 20 via sealing element 56 likewise in such a way as to substantially prevent liquid composition 22 from being contaminated with air and materials in air. These two operations can, for instance, be performed in the vacuum chamber directly after the operation of preparing liquid composition 22 in the chamber without removing any of the components of the container assembly of FIG. 1 from the chamber between any of the operations.

Dispenser 26 can be hermetically attached to container 20 via sealing element 56 prior to introducing liquid composition 22 and pressurant gas 24 into container 20 if composition 22 and gas 24 can be introduced into container 20 via dispenser 26. In that case, the hermetic sealing of dispenser 26 to container 20 can be performed at atmospheric pressure. Liquid composition 22 and pressurant gas 24 are subsequently introduced into container 20 via dispenser 26. If liquid composition 22 needs to be maintained highly pure, the introduction of composition 22 and pressurant gas 24 into container 20 is performed by placing the partially completed assembly of container 20, dispenser 26, and sealing element 56 in the vacuum chamber, introducing composition 22 into container 20 via dispenser 26 with the pressure in the chamber at the above-mentioned below-atmospheric conditions, and introducing gas 24 into container 20. The introduction of pressurant gas 24 into container 20 can be performed outside the vacuum chamber.

Material of liquid composition 22 is administered to a person sufficiently to treat a debilitating medical condition of the person or/and to prevent the person from contracting the debilitating medical condition. The administration is performed by using dispenser 26 to dispense that material from the container assembly of FIG. 1 and directing the dispensed material to the person. The administration of material of liquid composition 22 is variously performed topically, orally, intranasally, intraotically, vaginally, rectally, urethrally, and by injection as further described above. Injection administration can also be used by directing material of liquid composition 22 to an injection-administering tool such as a syringe. The debilitating medical condition can be any of the above-identified debilitating medical conditions, namely bacterial, eukaryotic, prion-caused, and viral infections, including fungal, spore-caused, and parasitic infections, and non-pathogenic-caused inflammation, again specifically including allergic rhinitis, arthritis, bronchitis, hemorrhoids, urticaria, toothache, tinea pedis, acute viral nasopharyngitis, herpes simplex, dandruff, itching, bromhidrosis, and vaginitis.

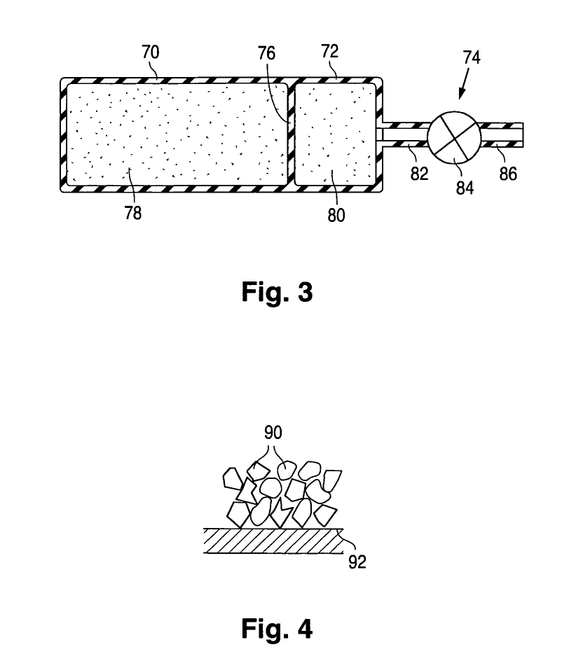

Another product in accordance with the invention is a double-container assembly as illustrated in FIG. 3 for controllably dispensing, or delivering, a liquid form of the medicinal drug of type I or II. The hardware components of the double-container assembly of FIG. 3 consist of a hermetically sealed first container 70, a second container 72, and a dispenser 74. Containers 70 and 72 form a container unit sharing a common breakable wall 76 which is designed to be broken by a user without significantly otherwise damaging the structural integrity of the double-container assembly. Container 70 contains a therapeutically inactive liquid carrier 78, normally water for the drug of type I or II. The water for liquid carrier 78 can be high-purity water such as deionized water and can include oxidative reductive potential water or/and super-oxidized water. Container 72 contains a primary powder composition 80 consisting at least partially of active material formed with the drug of type I or II.

Dispenser 74 consists of an inlet port 82, a valve 84, and an outlet port 86. Inlet port 82 is attached to, or integral with, one of containers 70 and 72 at a location spaced apart from breakable wall 76. In the example of FIG. 3, inlet port 82 is integral with, or attached to, container 72 that contains powder composition 80. Valve 84 controllably connects inlet port 82 and outlet port 86 so that material can move through dispenser 74 when valve 84 is at least partially open and is substantially prevented from moving through dispenser 74 when valve 84 is closed.

The double-container assembly of FIG. 3 can be provided to a user as a single unit in which dispenser 76 is attached to, or integral with, one of containers 70 and 72 as a result of inlet port 82 being attached to, or integral with, one of containers 70 and 72 so that. Alternatively, dispenser 74 can be provided to a user as a separate unit from containers 70 and 72. In that case, dispenser 74 is configured so as to be suitable for attachment to container 72 containing powder composition 80. More particularly, container 72 normally has a location specifically configured to receive inlet port 82. Connection of dispenser 74 to container 72 in this alternative is preferably done shortly before dispensing any of the liquid form of the medicinal drug of type I or II from the double-container assembly of FIG. 3.

When the active material in powder composition 80 is implemented with the medicinal drug of type I, the active material contains salt of peroxymonosulfuric acid, preferably potassium hydrogen peroxymonosulfate. The active material in the implementation of composition 80 using the drug of type I may include one or more of inorganic halide, metal phosphate, sulfamic acid, a non-reducing organic acid, and a surfactant as additional materials. In the situation where the active material in powder composition 80 is implemented with the medicinal drug of type II, the active material contains inorganic halide an oxidizing agent reactable in water with the inorganic halide to generate hypohalite ions. The active material in the implementation of composition 80 using the drug of type II may include one or more of metal phosphate, sulfamic acid, a non-reducing organic acid, and a surfactant as additional materials. All of these additional materials, except possibly the surfactant, are normally present in each implementation of powder composition 80. Candidates for the inorganic halide, the metal phosphate, the non-reducing acid, and the surfactant in either implementation of composition 80 and for the oxidizing agent in the implementation using the drug of type II are presented above.

The double-container assembly of FIG. 3 is manufactured in generally the following way. Dispenser 74, the container unit consisting of containers 70 and 72, and powder composition 80 are separately fabricated. The container unit is manufactured so that each of containers 70 and 72 has an opening. If the double-container assembly is provided to a user as a single unit, dispenser 74 is attached to container 72, preferably in a hermetic manner. Liquid carrier 78 is separately provided.

Regardless of whether the double-container assembly of FIG. 3 is provided to a user as a single unit or dispenser 74 is provided to a user as a separate unit from the container unit formed with containers 70 and 72, liquid carrier 78 is introduced into container 70 via its opening. The opening in container 70 is then closed and hermetically sealed. Powder composition 80, which is created by combining powders of the components of the medicinal drug of type I or II, is similarly introduced into container 72 via its opening. The opening in container 72 is similarly closed and sealed, preferably hermetically, to complete the fabrication process. The introduction of powder composition 80 into container 72 and the closure of its opening can be performed before or after introducing liquid carrier 78 into container 70 and closing its opening.

A user uses the double-container assembly of FIG. 3 in the following way. If dispenser 74 is provided to a user as a separate unit from the container unit formed with containers 70 and 72, the user attaches dispenser 74 to container 72. Regardless of whether the double-container assembly is provided to the user as a single unit or dispenser 74 is provided to the user as a separate unit from the container unit formed with containers 70 and 72, the user now breaks common wall 76 by suitably pressing on wall 76 to form one or more openings in wall 76 without significantly otherwise damaging the structural integrity of the double-container assembly. The breakage of wall 76 enables the respective internal volumes of containers 70 and 72 to be connected together. Liquid carrier 78 and powder composition 80 thereby combine to form a further liquid composition containing the medicinal drug of type I or II. The user typically shakes the double-container assembly so as to mix powder composition 80 into liquid carrier 78 so that the active material is dispersed largely throughout carrier 78.

Material of the further liquid composition containing the medicinal drug of type I or II in the double-container assembly of FIG. 3 is administered to a person sufficiently to treat a debilitating medical condition of the person or/and to prevent the person from contracting the debilitating medical condition. The administration is performed by using dispenser 74 to dispensing that material from the double-container assembly and directing the dispensed material to the person. Material of the further liquid composition is variously administered topically, orally, intranasally, intraotically, vaginally, rectally, and urethrally as further described above. Injection administration can also be used by directing material of the further liquid composition to an injection-administering tool such as a syringe. The debilitating medical condition can again be any of the above-identified debilitating medical conditions, namely bacterial, eukaryotic, prion-caused, and viral infections, including fungal, spore-caused, and parasitic infections, and non-pathogenic-caused inflammation and specifically including allergic rhinitis, arthritis, bronchitis, hemorrhoids, urticaria, toothache, tinea pedis, acute viral nasopharyngitis, herpes simplex, dandruff, itching, bromhidrosis, and vaginitis.

Importantly, powder composition 80 has a long shelf life. The further liquid composition need not be formed from liquid carrier 78 and powder composition 80 until shortly before the further liquid composition is administered to a person. This enables the double-container assembly of FIG. 3 to have a long shelf life.

A further product in accordance with the invention is a powder composition formed with solid particles 90 as generally depicted in FIG. 4. Solid particles 90 are illustrated in FIG. 4 as overlying a surface 92 which can, for example, be the bottom of a container (not shown). The shapes of particles 90 can be generally the same or can vary significantly. For instance, particles 90 can be of generally random shapes. The sizes of particles 90 can also vary significantly. Particles 90 consist of solid active material and solid particle support material which enables particles 90 to achieve the concentration characteristics described below and which serves as a solid carrier for the active material. Because particles 90 consist of solid material, the powder composition of FIG. 4 has a long shelf life.

The active material in solid particles 90 consists of the medicinal drug of type I in one implementation of the powder composition. The active material in this implementation of the powder composition thereby includes salt of peroxymonosulfuric acid, preferably potassium hydrogen peroxymonosulfate. The drug of type I is present at a sufficiently low concentration in particles 90 in this implementation of the powder composition due to the presence of the particle support material that the average mass percentage of the salt of peroxymonosulfuric acid in particles 90 is no more than 10%, preferably no more than 5%, more preferably no more than 2%, even more preferably no more than 1%. That is, the mass of the salt of peroxymonosulfuric acid is no more than 10%, preferably no more than 5%, more preferably no more than 2%, even more preferably no more than 1%, of the mass of particles 90. The support material is normally substantially chemically non-reactive with the salt of peroxymonosulfuric acid and with reaction product of the salt of peroxymonosulfuric acid and any other material in this implementation of the powder composition when it is dry or combined with water.

The active material in particles 90 in the implementation of the powder composition containing the medicinal drug of type I may include one or more of inorganic halide, metal phosphate, sulfamic acid, a non-reducing organic acid, and a surfactant as additional materials. The support material is then normally substantially chemically non-reactive with each additional material and with reaction product of each additional material and other material in this implementation of the powder composition when it is dry or combined with water.

In another implementation of the powder composition, the active material in solid particles 90 consists of the medicinal drug of type II. Taking note that the drug of type II includes inorganic halide and an oxidizing agent reactable in water with the inorganic halide to generate hypohalite ions, the drug of type II is present at a sufficiently low concentration in particles 90 of this second implementation of the powder composition due similarly to the presence of the particle support material that the average mass percentage of the oxidizing agent in particles 90 is no more than 10%, preferably no more than 5%, more preferably no more than 2%, even more preferably no more than 1%. In other words, the mass of the oxidizing agent is no more than 10%, preferably no more than 5%, more preferably no more than 2%, even more preferably no more than 1%, of the mass of particles 90. The support material is normally substantially chemically non-reactive with the oxidizing agent and with reaction product of the oxidizing agent and other material in the second implementation of the powder composition when it is dry or combined with water.

The active material in particles 90 of the powder containing the medicinal drug of type II may include one or more of metal phosphate, sulfamic acid, a non-reducing organic acid, and a surfactant as additional materials. The support material is then similarly normally substantially chemically non-reactive with each additional material and with reaction product of each additional material and other material in the second implementation of the powder composition when it is dry or combined with water.

All of the preceding additional materials, except possibly the surfactant, are normally present in each implementation of the powder composition formed with solid particles 90. Candidates for the inorganic halide, the metal phosphate, the non-reducing acid, and the surfactant in either implementation of the powder composition and for the oxidizing agent in the implementation using the medicinal drug of type II are presented above.

The average diameter of particles 90 of the powder containing the medicinal drug of type I or II is normally no more than 500 μm, preferably no more than 400 μm, more preferably no more than 300 μm. The average diameter of these powder particles is normally at least 50 μm, preferably at least 100 μm, more preferably at least 150 μm.

The particle-containing composition is prepared in the following manner. The active material and the particle support material are combined to form solid particles 90. This may involve combining the active material and the support material to form one or more relatively large solid pieces of material composed of the active and support materials and then cutting each such large solid piece up to form particles 90.

Solid particles 90 are combined with a therapeutically inactive liquid carrier, normally water, to produce a further medicinal composition as a liquid or semiliquid form of the medicinal drug of type I or II. The liquid carrier can be high-purity water such as deionized water and can include oxidative reductive potential water or/and super-oxidized water. The support material in particles 90 is normally substantially chemically non-reactive with the carrier. Whether the further medicinal composition is a liquid or a semiliquid depends primarily on the dynamic viscosity g. The further medicinal composition is typically deemed a liquid when dynamic viscosity g of the composition is less than 5 Pa-s at 25° C. and as a semiliquid when viscosity g of the composition is at least 5 Pa-s at 25° C.

Combining solid particles 90 and the liquid carrier can be performed by introducing particles 90 into the carrier. Alternatively or/and additionally, the carrier can be poured or otherwise placed on particles 90. In any event, particles 90 disintegrate in the carrier. The material of particles 90 typically dissolves in the carrier to form a solution.

The medicinal drug of type I or II may undergo chemical reactions in the course of combining solid particles 90 and the liquid carrier. The active material is thereby converted into further active material dispersed largely throughout the carrier. These chemical reactions cause the further active material to differ from the original active material in particles 90.

In the implementation of the powder composition where solid particles 90 contain the medicinal drug of type I and thus contain salt of peroxymonosulfuric acid, the further active material includes the salt of peroxymonosulfuric acid or/and reaction product of the salt of peroxymonosulfuric acid and other material of the powder composition containing the drug of type I. When particles 90 containing the drug of type I include one or more of inorganic halide, metal phosphate, sulfamic acid, a non-reducing organic acid, and a surfactant as additional materials, the further active material also includes each such additional material or/and reaction product of that additional material and other material of the powder composition containing the drug of type I.

The liquid carrier and particles 90 containing the medicinal drug of type I are combined at a percentage ratio of no more than 10% by mass, preferably no more than 5% by mass, more preferably no more than 2% by mass, of the salt of peroxymonosulfuric acid to the carrier. That is, the mass of the salt of peroxymonosulfuric acid is no more than 10%, preferably no more than 5%, more preferably no more than 2%, of the mass of the liquid carrier.

In the implementation of the powder composition where solid particles 90 contain the medicinal drug of type II and therefore an oxidizing agent and inorganic halide, the further active material includes the oxidizing agent or/and (ii) reaction product of the oxidizing agent and other material of the powder composition containing the drug of type II. When particles 90 containing the drug of type II include one or more of metal phosphate, sulfamic acid, a non-reducing organic acid, and a surfactant as additional materials, the further active material also includes each such additional material or/and reaction product of that additional material and other material of the powder composition containing the drug of type II.

The liquid carrier and particles 90 containing the medicinal drug of type II are combined at a percentage ratio of no more than 10% by mass, preferably no more than 5% by mass, more preferably no more than 2% by mass, of the oxidizing agent to the carrier. In other words, the mass of the oxidizing agent is no more than 10%, preferably no more than 5%, more preferably no more than 2%, of the mass of the liquid carrier.

The support material in solid particles 90 is, as indicated above, normally substantially chemically non-reactive with the liquid carrier. The particle support material is, as also indicated above, normally substantially chemically non-reactive with the components of the medicinal drug of type I or II and with reaction products of the components of the drug of type I or II when the powder containing the drug of type I or II is dry or introduced into water. In cases where the carrier does not consist of water, the particle support material and the carrier can be chosen so that the particle support material is substantially chemically non-reactive with the components of the medicinal drug of type I or II and with reaction products of the components of the drug of type I or II when the powder containing the drug of type I or II is dry or introduced into the carrier. However, the liquid nature of the carrier, especially when it consists of water, promotes chemical reactions among the components of the drug of type I or II and between the components of the drug of type I or II and the carrier.

Utilizing the particle support material and the liquid carrier in accordance with the formulation ranges presented above effectively replaces some of the carrier with the particle support material. Since the liquid carrier promotes chemical reactions involving the components of the medicinal drug of type I or II and since the particle support material and the carrier can be chosen so that the particle support material is substantially chemically non-reactive in dry form and in the presence of the carrier, the result of so replacing some of the carrier with the particle support material is to produce fewer chemical reactions in the further medicinal composition formed by combining solid particles 90 and the carrier than in the above-mentioned liquid medicinal composition formed by combining the drug of type I or II with a water carrier. The incidence of physical/chemical phenomena which cause the potency of the drug of type I or II in the further composition to decrease with time is therefore expected to decrease. The further medicinal composition is therefore expected to maintain its potency at an adequate level for increased time.

Material of the further liquid or semiliquid medicinal composition formed by combining solid particles 90 and the liquid carrier is administered to a person sufficiently to treat a debilitating medical condition of the person or/and to prevent the person from contracting the debilitating medical condition. The administration of material of the further liquid or semiliquid composition is variously performed topically, orally, intranasally, intraotically, vaginally, rectally, urethrally, and by injection as further described above depending on whether the further medicinal composition is a liquid or a semiliquid. The debilitating medical condition can once again be any of the above-identified debilitating medical conditions, namely bacterial, eukaryotic, prion-caused, and viral infections, including fungal, spore-caused, and parasitic infections, and non-pathogenic-caused inflammation and specifically including allergic rhinitis, arthritis, bronchitis, hemorrhoids, urticaria, toothache, tinea pedis, acute viral nasopharyngitis, herpes simplex, dandruff, itching, bromhidrosis, and vaginitis.

Another product in accordance with the invention is a composition containing a liquid carrier, active material dispersed largely throughout the carrier, and an inhibitor dispersed largely throughout the liquid carrier. The active material in the inhibitor-containing composition consists of the medicinal drug of type I or II. More specifically, the active material includes an oxidizing agent or/and reaction product of the oxidizing agent and other material of the composition. The oxidizing agent contains active oxygen consisting of chemically readily transferable oxygen atoms. Each molecule of the oxidizing agent has at least one atom of active oxygen. The inhibitor inhibits the composition from losing active oxygen. This enables the potency of the inhibitor-containing composition to be maintained at a suitably high level for an extended time so that the composition has increased shelf life.

The capability of the inhibitor to prevent active oxygen from being lost from the inhibitor-containing composition is described with reference to a comparative composition which lacks the inhibitor but is otherwise constituted the same as the inhibitor-containing composition. The inhibitor causes the loss of active oxygen from the inhibitor-containing composition to be at least 10% lower, preferably at least 20% lower, more preferably at least 30% lower, as a function of time than what would arise in the comparative composition up to the point at which the comparative composition has lost 50% of its active oxygen.

When the active material in the inhibitor-containing composition is implemented with the medicinal drug of type I, the active material contains salt of peroxymonosulfuric acid or/and reaction product of the salt of peroxymonosulfuric acid and other material of the composition. The salt of peroxymonosulfuric acid preferably is potassium hydrogen peroxymonosulfate. Each molecule of the salt of peroxymonosulfuric acid has one atom of active oxygen for each single oxygen-oxygen bond in the molecule and thus one atom of active oxygen for each SO₅ group in the molecule. The active material in the implementation of the inhibitor-containing composition using the drug of type I may include one or more of the following additional materials: (a) inorganic halide or/and reaction product of the inorganic halide and other material of the composition, (b) metal phosphate or/and reaction product of the metal phosphate and other material of the composition, (c) sulfamic acid or/and reaction product of sulfamic acid and other material of the composition, (d) a non-reducing organic acid or/and reaction product of the organic acid and other material of the composition, and (e) a surfactant or/and reaction product of the surfactant and other material of the composition.

In the situation where the active material in the inhibitor-containing composition is implemented with the medicinal drug of type II, the liquid carrier consists of water, and the active material contains inorganic halide or/and reaction product of the inorganic halide and other material of the composition in addition to the oxidizing agent or/and reaction product of the oxidizing agent and other material of the composition. The oxidizing agent is reactable in water with the inorganic halide to generate hypohalite ions. The active material in the implementation of the inhibitor-containing composition using the drug of type II may include one or more of the following additional materials: (a) metal phosphate or/and reaction product of the metal phosphate and other material of the composition, (b) sulfamic acid or/and reaction product of sulfamic acid and other material of the composition, (c) a non-reducing organic acid or/and reaction product of the organic acid and other material of the composition, and (d) a surfactant or/and reaction product of the surfactant and other material of the composition.

The above-mentioned reaction products are variously present in the inhibitor-containing composition implemented with the medicinal drug of type I or II due to combining the drug of type I or II with the liquid carrier. All of the above-mentioned additional materials, except possibly the surfactant, are normally present in each implementation of the inhibitor-containing composition. Candidates for the inorganic halide, the metal phosphate, the non-reducing acid, and the surfactant in either implementation of the inhibitor-containing composition and for the oxidizing agent in the implementation using the drug of type II are presented above.

The inhibitor-containing composition is prepared by combining the active material, the liquid carrier, and the inhibitor so that the active material and the inhibitor are dispersed largely throughout the carrier. For example, the active material and the inhibitor can be introduced into the carrier. Alternatively or/and additionally, the carrier can be poured on or otherwise placed on the active material or/and the inhibitor. In either case, suitable mixing of the active material, the inhibitor, and the carrier is typically performed to enable the active material and the inhibitor to be dispersed largely throughout the carrier.

Water, preferably deionized water, normally serves as the liquid carrier in the inhibitor-containing composition. For the preferred case in which the liquid carrier consists of deionized water, the deionized water has the above-mentioned conductivity and resistivity characteristics prior to combining the deionized water, the active material, and the inhibitor to form the inhibitor-containing composition. The liquid carrier can include oxidative reductive potential water or/and super-oxidized water.

Material of the inhibitor-containing composition is administered to a person sufficiently to treat a debilitating medical condition of the person or/and to prevent the person from contracting the debilitating medical condition. The administration of material of the inhibitor-containing composition is variously performed topically, orally, intranasally, intraotically, vaginally, rectally, urethrally, and by injection as further described above. The debilitating medical condition can again be any of the above-identified debilitating medical conditions, namely bacterial, eukaryotic, prion-caused, and viral infections, including fungal, spore-caused, and parasitic infections, and non-pathogenic-caused inflammation and specifically including allergic rhinitis, arthritis, bronchitis, hemorrhoids, urticaria, toothache, tinea pedis, acute viral nasopharyngitis, herpes simplex, dandruff, itching, bromhidrosis, and vaginitis.

While the invention has been described with reference to preferred embodiments, this description is solely for the purpose of illustration and is not to be construed as limiting the scope of the invention claimed below. For instance, the disintegrable composition containing active material formed with the medicinal drug of type I or II need not be fully solid provided that pieces of the disintegrable composition substantially maintain their shape as time passes prior to introduction of the composition into the liquid in which the composition disintegrates. More particularly, the disintegrable composition can be a highly viscous semiliquid, e.g., a semiliquid of similar dynamic viscosity to that of glass.

Instead of having common wall 76 in the double-container assembly of FIG. 3 be breakable, dispenser 74 can be modified to have inlet ports connected to both of containers 70 and 72 so that matter of liquid carrier 78 and powder composition 80 separately enter dispenser 74. Containers 70 and 72 can even be physically separate from each other. Container 70 that contains liquid carrier 78 can be configured in the same way as container 20 in the container assembly of FIG. 1. In any event, dispenser 74 is also modified so as to combine the incoming matters of liquid carrier 78 and powder composition 80 by suitably mixing that incoming material to produce the liquid composition provided from outlet port 86. As in the double-container assembly of FIG. 3, the further liquid composition need not be formed from liquid carrier 78 and powder composition 80 until shortly before the further liquid composition is administered to a person. This variation of the double-container assembly of FIG. 3 likewise has a long shelf life.

The above-mentioned oxidizing agents which release oxygen may be replaced, in forming variations of some embodiments of the medicinal drug of type II, with oxidizing agents which do not release oxygen but which accept electrons in reduction-oxidization chemical reactions at oxidizing strength roughly equivalent to the oxidizing agents which release oxygen. The products of the invention can be used to treat non-human animals variously inflicted with diseases and other debilitating medical conditions caused by bacterial, eukaryotic, prion, and viral pathogens, including fungal, spore, and parasitic pathogens, and non-pathogenic inflammation. The products of the invention can be used to prevent non-human animals from contracting such debilitating medical conditions.

In addition to successfully treating, and preventing the occurrence of, allergic rhinitis, arthritis, bronchitis, hemorrhoids, urticaria, toothache, tinea pedis, acute viral nasopharyngitis, herpes simplex, dandruff, itching, bromhidrosis, and vaginitis in humans, the products of the invention can be used to treat or/and prevent many other debilitating human health conditions. Such other debilitating human health conditions may, for example, include medical conditions dealing with (a) the circulatory and cardiovascular system involved in pumping and circulating blood through the body in its organs and tissues, including blood vessels, arteries, capillaries, heart, and veins, (b) the digestive or gastrointestinal system involved in the ingestion and digestion of food with or through the salivary glands, esophagus, stomach, liver, gallbladder, pancreas, appendix, intestines, rectum, and anus, (c) the endocrine system which chemically controls various functions of cells, tissues, and organs through the secretion of hormones made by endocrine glands, such as the hypothalamus, pituitary, pineal, thyroid, parathyroid, and adrenal gland as well as the islets of Langerhans, ovaries, pancreas, and testes, (d) the integumentary system consisting of the skin and its related structures, the hair, nails, sweat glands, and sebaceous glands, (e) the endocannabinoid system in which the neuromodulatory lipids and receptors involve a variety of physiological processes of the brain, including, appetite, cognition, emotional responses, homeostasis, motor learning, pain-sensation, and synaptic plasticity, (f) the immune system which neutralizes pathogenic organisms or/and foreign matter and which includes organs, such as the skin and mucous membranes, adenoids, antibody producers, leukocytes, lymph nodes, and lymphoid tissue (as in the gastrointestinal tract and bone marrow), lymphocytes including B cells and T cells, stem cells, spleen, thymus, and tonsils, (g) the lymphatic system which is involved in the circulation of lymph between the cells, tissues, and organs to the blood stream and which includes tonsils, thymus, spleen, lymph, lymph nodes, lymphatic vessels, lymphocytes, sinuses through which lymph is carried, lymphoid tissues, and bone marrow where stem cells differentiate into precursors of B cells and T cells, and (h) the muscular system formed with muscle cells and tissues that brings about movement of organs, other body parts, maintenance of posture, and heat production. The muscular system includes three basic kinds of muscles, namely (h1) the cardiac muscles which form the walls of the heart, (h2) the smooth muscles which are found in the internal organs and assist in the involuntary movements that occur in the circulatory, digestive, excretory, reproductive, and respiratory systems, and (h3) the skeletal muscles which are attached to the bones and enable voluntary movement of limbs.

Such other debilitating human health conditions may, for example, further include medical conditions dealing with (i) the musculoskeletal system in which the skeleton, muscles, bones, cartilage, joints, ligaments, tendons, and associated tissues provide movements to the body and maintain its structural form, (j) the nervous system in which the bodily system of cells, neurons, tissues, and organs regulates (collects, transfers, and processes) the body's function to internal and external stimuli and transmits impulses to the effector organs and also regulates secretions of the endocrine system by the action of neurohormones and which includes the brain, spinal cord, peripheral and autonomic nerves, nerves, ganglia, parts of the receptor organs, parts of the effector organs, and the sensory organs such as the ears and eyes, (k) the reproductive system in which organs and parts function in reproduction, the female includes, ovaries, fallopian tubes, uterus, cervix, vagina, vulva, and also the mammary glands, and in the male includes, seminal vesicles, prostate, urethra, vas deferens, testes, and penis, (l) the respiratory system which is involved in the intake and exchange of oxygen and carbon dioxide between the body and the environment and which includes the nose, nasal passages, pharynx, larynx, trachea, bronchi, heart, ribs, diaphragm, and lungs, (m) the skeletal system formed with bones, cartilage, joints, tendons, and other connective tissues which protect and support the body tissues, internal organs and produces blood cells and stores minerals, (n) the urinary system which is formed with the kidneys, ureters, bladder, and urethra of the urinary tract and which is involved in the regulation of water content and electrolyte concentration through the excretion of metabolic wastes, excess water, and electrolytes in the form of urine, and (o) the vestibular system which is involved with the equilibrium and organs mediating the labyrinthine sense and which includes the anterior canal, utricle, saccule, nerve, cochlea, horizontal canal, and posterior canal.

More specifically, the products of the invention may be used to treat or/and prevent (i) bronchial asthma, (ii) tuberculosis, (iii) cholera, (iv) syphilis, (v) meningitis, (vi) pneumonia, (vii) sepsis, (viii) cystic fibrosis, (ix) aspergillosis, (x) psoriasis, (xi) aspergilloma, (xii) amoebiasis, (xiii) Lyme disease, (xiv) malaria, (xv) prion infectious diseases including transmissible spongiform encephalopathic diseases such as Creutzfeldt-Jakob disease, Gerstmann-Sträussler-Scheinker syndrome, fatal familial insomnia, and kuru, (xvi) Alpers' syndrome, (xvii) AIDS, (xviii) hepatitides including hepatitis A, hepatitis B, hepatitis C, hepatitis D, and hepatitis E, (xix) cancers including carcinoma, sarcoma, leukemia, lymphoma, germ cell tumors, and blastic tumors, (xx) heart diseases and conditions including arrhythmias, cardiomyopathies, cardiovascular diseases, congenital heart defects, heart infections, and valvular heart diseases, (xxi) hypercholesterolemia, (xxii) hypertriglyceridemia, (xxiii) atherosclerosis, (xxiv) diabetes mellitus including diabetes type 1, diabetes type 2, gestational diabetes, congenital diabetes, cystic fibrosis-related diabetes, steroid diabetes, and forms of monogenic diabetes, (xxv) ocular disorders including conjunctivitis, trachoma, and uveitis, (xxvi) ear disorders including Meniere's disease, tinnitus, and otitis media, and (xxvii) urethral disorders including urethral stricture and urethritis.

Furthermore, the products of the invention may be used to treat or/and prevent (i) blast wounds, (ii) dermatitis including contact dermatitis, atopic dermatitis, dermatitis herpetiformis, seborrheic dermatitis, nummular dermatitis, and stasis dermatitis, (iii) diabetic ulcers, (iv) gastroenteritis, (v) helminthiasis including soil-transmitted helminthes, ancylostomiasis, ascariasis, filariasis, onchocerciasis, schistosomiasis, and trichuriasis, (vi) human papillomavirus, (vii) influenza, and (viii) mother to child transmission of chlamydia, gonorrhea, hepatitis, HIV, human papillomavirus, and tuberculosis.

The products of the invention may be used to treat or/and prevent virally caused debilitating human medical conditions arising, for example, from: (i) double-stranded DNA viruses including caudovirales, herpesvirales, ascoviridae, adenoviridae, asfarviridae, baculoviridae, coccolithoviridae, corticoviridae, fuselloviridae, guttaviridae, iridoviridae, lipothrixviridae, mimiviridae, nimaviridae, papillomaviridae, phycodnaviridae, plasmaviridae, polyomaviridae, poxyiridae, rudiviridae, tectiviridae, ampullavirus, nudivirus, salterprovirus, sputnik virophage, and rhizidiovirus, (ii) single-stranded DNA viruses including the bacteriophage families inoviridae and microviridae and anelloviridae, circoviridae, geminiviridae, nanoviridae, and parvoviridae, (iii) double-stranded RNA viruses including, birnaviridae, cystoviridae, hypoviridae, partitiviridae, reoviridae, and totiviridae, (iv) positive-sense single-stranded RNA viruses including, nidovirales, picornavirales, tymovirales, astroviridae, barnaviridae, bromoviridae, caliciviridae, closteroviridae, flaviviridae, leviviridae, luteoviridae, narnaviridae, nodaviridae, potyviridae, tetraviridae, togaviridae, tombusviridae, benyvirus, furovirus, hepevirus, hordeivirus, idaeovirus, ourmiavirus, pecluvirus, pomovirus, sobemovirus, tobamovirus, tobravirus, and umbravirus, (v) negative-sense single-stranded RNA viruses including, mononegavirales, arenaviridae, bunyaviridae, orthomyxoviridae, deltavirus, nyavirus, ophiovirus, tenuivirus, and varicosavirus, (vi) single-stranded RNA reverse-transcription viruses including, retroviruses, and (vii) double-stranded DNA reverse-transcription viruses including, hepadnaviridae.

The products of the invention may be used to treat or/and prevent bacterially caused debilitating human medical conditions arising, for example, from: (i) gram positive bacteria with no outer membrane including, actinobacteria, firmicutes, and tenericutes, (ii) gram negative bacteria with outer membrane including, aquificae, bacteroidetes, chlamydiae, chlorobi, deinococcus-thermus, fusobacteria, gemmatimonadetes, nitrospirae, proteobacteria, spirochaetes, synergistetes, and verrucomicrobia, and (iii) acidobacteria, chloroflexi, chrysiogenetes, cyanobacteria, deferribacteres, dictyoglomi, fibrobacteres, planctomycetes, thermodesulfobacteria, and thermotogae.

The products of the invention may be used to treat or/and prevent fungal caused debilitating human medical conditions arising, for example, from blastocladiomycota, chytridiomycota, glomeromycota, microsporidia, neocallimastigomycota, dikarya, zygomycota, and deuteromycota.

The products of the invention may be used to treat or/and prevent parasite-caused debilitating human medical conditions arising, for example, from: (i) endoparasites including the plant group of rafflesiaceae, (ii) parasitic worms including the groups of cestodes, nematodes, and trematodes, (iii) ectoparasites including the plant groups of broomrape, cuscuta, mistletoe, santalum, toothwort, and wood rose, (iv) protists in the group of bikonts including apusozoa, archaeplastida, excavata, centrohelida, chromalveolata, and rhizaria, (v) protists in the group of unikonta including amoebozoa and opisthokonta, and (vi) the metazoa group including eumetazoa, placozoa, and porifera.

The products of the invention may be used to treat or/and prevent non-pathogenic inflammatory caused debilitating human medical conditions arising, for example, from chronic fibrinous, granulomatous, pseudomembranous, purulent, serous, or/and ulcerative inflammation.

The compositions provided by the products of the invention, including or/and along with compositions having the formulations of the medicinal drug of types I and II, may be used as anti-pathogenic, anti-inflammatory, or/and as disease preventative materials to treat, or otherwise be applied to, nutrients, oils, eukaryotic products, animal food products including meat and seafood, crops, eggs, flour, honey, mushrooms, seasonings, seeds, sugar, vegetables, fruits and dry fruits, dairy products, beverage products, cereals, other food products, or/and their derived products.

The compositions provided by the products of the invention, including or/and along with compositions having the formulations of the medicinal drug of types I and II, may be used as (i) algaecides, algaestats, bleaches, degreasers, deodorants, deodorizers, detergents and soaps, emulsifiers, insecticides and pesticides, sanitizers, stain removers, whiteners, and disinfectants for inanimate surfaces including asphalt, ceramic, clay, concrete, contact lens, drywall, glass, granite, marble, metal, plastic, salt, sand, slate, stone, and wood, for animate surfaces including body, face, hands, and feet, for blood, and for airborne pathogens, (ii) etchants for various materials including metals, and (iii) oxidizers for various materials including coal, gases, metals, metalloids, nonmetals, oils, paper, paper pulp, wood, and wood pulp.

The compositions provided by the products of the invention, including or/and along with compositions having the formulations of the medicinal drug of types I and II, may be used in bandages, bark, burn treatment materials, cosmetic products, dentures, diapers, fertilizer, fibers including textiles from animal (wool and silk), plant (cotton, flax, and jute), mineral (asbestos and glass fiber), other natural, and synthetic (nylon, polyester, and acrylic, cellulose, and polymer) sources, microfibers, leather, rubber, hair products, handheld wands, mops and scrubbing products, industrial products, medicinal products, preservation and other shelf-life-increasing products, sanitary wipes, soil, tampons, toothpaste and mouthwash, wound treatment materials, and other consumer products.

In addition to the previous applications, the products of the invention can be used in other non-medical industrial and commercial applications. Furthermore, the products of the invention can be used to treat conditions other than human or non-human animal debilitating medical conditions such as colony collapse disorder of bees. Various changes and applications may thus be made without departing from the true scope of the invention as defined in the appended claims.

We claim:

1. A disintegrable composition for introduction into a liquid, the composition (a) having a mass of an initial mass value immediately prior to introduction into the liquid, (b) comprising active material comprising salt of peroxymonosulfuric acid, and (c) being of such a structure that the composition gradually disintegrates upon introduction into the liquid for enabling particles of the composition to disperse into the liquid so that the mass of the composition is 10% to 90% of the initial mass value 1 hour to 100 days after the composition is introduced into the liquid.

2. A composition as in claim 1 wherein the salt of peroxymonosulfuric acid comprises potassium hydrogen peroxymonosulfate.

3. A composition as in claim 1 wherein the active material further includes at least one of the following additional materials: inorganic halide, metal phosphate, sulfamic acid, a non-reducing organic acid, and a surfactant.

4. A method comprising:
combining material of the composition of claim 1 with material of the liquid to form a further composition; and
administering material of the further composition to a person sufficiently to treat a medical condition of the person or/and to prevent the person from contracting the medical condition.

5. A disintegrable composition for introduction into a liquid comprising water, the composition (a) having a mass of an initial mass value immediately prior to introduction into the liquid, (b) comprising active material comprising inorganic halide and an oxidizing agent reactable in water with the inorganic halide to generate hypohalite ions, and (c) being of such a structure that the composition gradually disintegrates upon introduction into the liquid for enabling particles of the composition to disperse into the liquid so that the mass of the composition is 10% to 90% of the initial mass value 1 hour to 100 days after the composition is introduced into the liquid.

6. A composition as in claim 5 wherein the active material further includes at least one of the following additional materials: metal phosphate, sulfamic acid, a non-reducing organic acid, and a surfactant.

7. A method comprising:
combining material of the composition of claim 5 with material of the liquid to form a further composition; and
administering material of the further composition to a person sufficiently to treat a medical condition of the person or/and to prevent the person from contracting the medical condition.

8. A composition comprising:
a carrier; and
active material dispersed largely throughout the carrier such that the composition is semiliquid, the active material comprising salt of peroxymonosulfuric acid or/and reaction product of the salt of peroxymonosulfuric acid and other material of the composition.

9. A composition as in claim 8 wherein the salt of peroxymonosulfuric acid comprises potassium hydrogen peroxymonosulfate.

10. A composition as in claim 8 wherein the active material further includes at least one of the following additional materials:
inorganic halide or/and reaction product of the inorganic halide and other material of the composition;
metal phosphate or/and reaction product of the metal phosphate and other material of the composition;
sulfamic acid or/and reaction product of sulfamic acid and other material of the composition;
a non-reducing organic acid or/and reaction product of the organic acid and other material of the composition; and
a surfactant or/and reaction product of the surfactant and other material of the composition.

11. A method comprising administering material of the composition of claim 8 to a person sufficiently to treat a medical condition of the person or/and to prevent the person from contracting the medical condition.

12. A composition comprising:
a carrier; and
active material dispersed largely throughout the carrier such that the composition is semiliquid, the active material comprising (a) inorganic halide or/and reaction product of the inorganic halide and other material of the composition and (b) an oxidizing agent or/and reaction product of the oxidizing agent and other material of the composition, the oxidizing agent being reactable in water with the inorganic halide to generate hypohalite ions.

13. A composition as in claim 12 wherein the active material further includes at least one of the following additional materials:
metal phosphate or/and reaction product of the metal phosphate and other material of the composition;
sulfamic acid or/and reaction product of sulfamic acid and other material of the composition;
a non-reducing organic acid or/and reaction product of the organic acid and other material of the composition; and
a surfactant or/and reaction product of the surfactant and other material of the composition.

14. A method comprising administering material of the composition of claim 12 to a person sufficiently to treat a medical condition of the person or/and to prevent the person from contracting the medical condition.

15. A product comprising:
a composition comprising a carrier and active material dispersed largely throughout the carrier, the active material comprising salt of peroxymonosulfuric acid or/and reaction product of the salt of peroxymonosulfuric acid and other material of the composition; and
a container containing the composition and substantially blocking transmission of visible light incident on the container from outside the container.

16. A product as in claim 15 wherein the salt of peroxymonosulfuric acid comprises potassium hydrogen peroxymonosulfate.

17. A product as in claim 15 wherein the active material further includes at least one of the following additional materials:
inorganic halide or/and reaction product of the inorganic halide and other material of the composition;
metal phosphate or/and reaction product of the metal phosphate and other material of the composition;
sulfamic acid or/and reaction product of sulfamic acid and other material of the composition;
a non-reducing organic acid or/and reaction product of the organic acid and other material of the composition; and
a surfactant or/and reaction product of the surfactant and other material of the composition.

18. A method of manufacturing the product of claim 15, the method comprising:
providing the composition and the container; and
introducing the composition into the container.

19. A method comprising administering material of the composition of claim 15 to a person sufficiently to treat a medical condition of the person or/and to prevent the person from contracting the medical condition.

20. A product comprising:
a composition comprising a carrier and active material dispersed largely throughout the carrier, the active material comprising (a) inorganic halide or/and reaction product of the inorganic halide and other material of the composition and (b) an oxidizing agent or/and reaction product of the oxidizing agent and other material of the composition, the oxidizing agent being reactable in water with the inorganic halide to generate hypohalite ions; and
a container containing the composition and substantially blocking transmission of visible light incident on the container from outside the container.

21. A product as in claim 20 wherein the active material further includes at least one of the following additional materials:
metal phosphate or/and reaction product of the metal phosphate and other material of the composition;
sulfamic acid or/and reaction product of sulfamic acid and other material of the composition;
a non-reducing organic acid or/and reaction product of the organic acid and other material of the composition; and
a surfactant or/and reaction product of the surfactant and other material of the composition.

22. A method of manufacturing the product of claim 20, the method comprising:
providing the composition and the container; and
introducing the composition into the container.

23. A method comprising administering material of the composition of claim 20 to a person sufficiently to treat a medical condition of the person or/and to prevent the person from contracting the medical condition.

24. A product comprising:
a composition comprising a carrier and active material dispersed largely throughout the carrier, the active material comprising salt of peroxymonosulfuric acid or/and reaction product of the salt of peroxymonosulfuric acid and other material of the composition; and
a container which contains the composition and inside which the composition is subjected to an average pressure of more than 1 atm.

25. A product as in claim 24 wherein the salt of peroxymonosulfuric acid comprises potassium hydrogen peroxymonosulfate.

26. A product as in claim 24 wherein the active material further includes at least one of the following additional materials:
inorganic halide or/and reaction product of the inorganic halide and other material of the composition;
metal phosphate or/and reaction product of the metal phosphate and other material of the composition;
sulfamic acid or/and reaction product of sulfamic acid and other material of the composition;
a non-reducing organic acid or/and reaction product of the organic acid and other material of the composition; and
a surfactant or/and reaction product of the surfactant and other material of the composition.

27. A product as in claim 24 wherein the average pressure inside the container is at least 2 atm.

28. A method of manufacturing the product of claim 24, the method comprising:
providing the composition and the container; and
introducing the composition into the container.

29. A method comprising administering material of the composition of claim 24 to a person sufficiently to treat a medical condition of the person or/and to prevent the person from contracting the medical condition.

30. A product comprising:
a composition comprising a carrier and active material dispersed largely throughout the carrier, the active material comprising (a) inorganic halide or/and reaction product of the inorganic halide and other material of the composition and (b) an oxidizing agent or/and reaction product of the oxidizing agent and other material of the composition, the oxidizing agent being reactable in water with the inorganic halide to generate hypohalite ions; and
a container which contains the composition and inside which the composition is subjected to an average pressure of more than 1 atm.

31. A product as in claim 30 wherein the active material further includes at least one of the following additional materials:
metal phosphate or/and reaction product of the metal phosphate and other material of the composition;
sulfamic acid or/and reaction product of sulfamic acid and other material of the composition;
a non-reducing organic acid or/and reaction product of the organic acid and other material of the composition; and
a surfactant or/and reaction product of the surfactant and other material of the composition.

32. A product as in claim 30 wherein the average pressure inside the container is at least 2 atm.

33. A method of manufacturing the product of claim 30, the method comprising:
providing the composition and the container; and
introducing the composition into the container.

34. A method comprising administering material of the composition of claim 30 to a person sufficiently to treat a medical condition of the person or/and to prevent the person from contracting the medical condition.

35. A product comprising:
a composition comprising a carrier and active material dispersed largely throughout the carrier, the active material comprising salt of peroxymonosulfuric acid or/and reaction product of the salt of peroxymonosulfuric acid and other material of the composition;
a container which contains the composition; and
a dispenser hermetically attached to the container for controllably dispensing material of the composition from the container, the dispenser substantially preventing material outside the container and the dispenser from entering the container through the dispenser.

36. A product as in claim 35 wherein the salt of peroxymonosulfuric acid comprises potassium hydrogen peroxymonosulfate.

37. A product as in claim 25 wherein the active material further includes at least one of the following additional materials:
inorganic halide or/and reaction product of the inorganic halide and other material of the composition;
metal phosphate or/and reaction product of the metal phosphate and other material of the composition;
sulfamic acid or/and reaction product of sulfamic acid and other material of the composition;
a non-reducing organic acid or/and reaction product of the organic acid and other material of the composition; and
a surfactant or/and reaction product of the surfactant and other material of the composition.

38. A method of manufacturing the product of claim 35, the method comprising:
providing the composition, the container, and the dispenser; and
assembling the container, the dispenser, and the composition into a structure by a procedure that entails introducing the composition into the container.

39. A method comprising administering material of the composition of claim 35 to a person sufficiently to treat a medical condition of the person or/and to prevent the person from contracting the medical condition.

40. A product comprising:
a composition comprising a carrier and active material dispersed largely throughout the carrier, the active material comprising (a) inorganic halide or/and reaction product of the inorganic halide and other material of the composition and (b) an oxidizing agent or/and reaction product of the oxidizing agent and other material of the composition, the oxidizing agent being reactable in water with the inorganic halide to generate hypohalite ions;
a container which contains the composition; and
a dispenser hermetically attached to the container for controllably dispensing material of the composition from the container, the dispenser substantially preventing material outside the container and the dispenser from entering the container through the dispenser.

41. A product as in claim 40 wherein the active material further includes at least one of the following additional materials:
metal phosphate or/and reaction product of the metal phosphate and other material of the composition;
sulfamic acid or/and reaction product of sulfamic acid and other material of the composition;
a non-reducing organic acid or/and reaction product of the organic acid and other material of the composition; and
a surfactant or/and reaction product of the surfactant and other material of the composition.

42. A method of manufacturing the product of claim 40, the method comprising:
providing the composition, the container, and the dispenser; and
assembling the container, the dispenser, and the composition into a structure by a procedure that entails introducing the composition into the container.

43. A method comprising administering material of the composition of claim 40 to a person sufficiently to treat a medical condition of the person or/and to prevent the person from contracting the medical condition.

44. A product comprising:
a liquid carrier;
a first container containing the liquid carrier;
a primary composition comprising active material comprising salt of peroxymonosulfuric acid;
a second container containing the primary composition; and
a combining element attached to or integral with at least one of the containers, or suitable for attachment to at least one of the containers, for enabling matter of the primary composition and matter of the carrier to be controllably combined to form a further composition.

45. A product as in claim 44 wherein the salt of peroxymonosulfuric acid comprises potassium hydrogen peroxymonosulfate.

46. A product as in claim 44 wherein the active material further includes at least one of the following additional materials: inorganic halide, metal phosphate, sulfamic acid, a non-reducing organic acid, and a surfactant.

47. A method of manufacturing the product of claim 44, the method comprising:
providing the carrier, the composition, and the containers; and
assembling the containers, the carrier, and the composition into a structure by a procedure that entails introducing the carrier and the composition respectively into the first and second containers.

48. A method comprising administering material of the further composition of claim 44 to a person sufficiently to treat a medical condition of the person or/and to prevent the person from contracting the medical condition.

49. A product comprising:
a liquid carrier comprising water;
a first container containing the carrier;
a primary composition comprising active material comprising inorganic halide and an oxidizing agent reactable in the water with the inorganic halide to generate hypohalite ions; and
a second container containing the primary composition; and
a combining element attached to or integral with at least one of the containers, or suitable for attachment to at least one of the containers, for enabling matter of the primary composition and matter of the carrier to be controllably combined to form a further composition.

50. A product as in claim 49 wherein the active material further includes at least one of the following additional materials: metal phosphate, sulfamic acid, a non-reducing organic acid, and a surfactant.

51. A method of manufacturing the product of claim 49, the method comprising:
providing the carrier, the composition, and the containers; and
assembling the containers, the carrier, and the composition by a procedure that entails introducing the carrier and the composition respectively into the first and second containers.

52. A method comprising administering material of the further composition of claim 49 to a person sufficiently to treat a medical condition of the person or/and to prevent the person from contracting the medical condition.

53. A particle-containing composition comprising a plurality of solid particles comprised of support material and active material comprising salt of peroxymonosulfuric acid, the salt of peroxymonosulfuric acid being present in the particles at an average mass percentage of no more than 10%.

54. A composition as in claim 53 wherein the salt of peroxymonosulfuric acid comprises potassium hydrogen peroxymonosulfate.

55. A composition as in claim 53 wherein the particles are of an average diameter of no more than 500 μm.

56. A composition as in claim 53 wherein the active material includes at least one of the following additional materials: inorganic halide, metal phosphate, sulfamic acid, a non-reducing organic acid, and a surfactant.

57. A method of preparing the composition of claim 53, the method comprising combining the active material and the support material to form the particles.

58. A method comprising:
combining a liquid carrier and a composition as in claim 53 to produce a further composition in which further active material is dispersed largely throughout the carrier, the further active material comprising the salt of peroxymonosulfuric acid or/and reaction product of the salt of peroxymonosulfuric acid and other material of the further composition: and
administering material of the further composition to a person sufficiently to treat a medical condition of the person or/and to prevent the person from contracting the medical condition.

59. A particle-containing composition comprising a plurality of solid particles comprised of support material and active material comprising inorganic halide and an oxidizing agent reactable in water with the inorganic halide to generate hypohalite ions, the oxidizing agent being present in the particles at an average mass percentage of no more than 10%.

60. A composition as in claim 59 wherein the particles are of an average diameter of no more than 500 μm.

61. A composition as in claim 59 wherein the active material include at least one of the following additional materials: metal phosphate, sulfamic acid, a non-reducing organic acid, and a surfactant.

62. A method comprising:
combining a liquid carrier and a composition as in claim 59 to produce a further composition in which further active material is dispersed largely throughout the carrier, the further active material comprising the salt of peroxymonosulfuric acid or/and reaction product of the salt of peroxymonosulfuric acid and other material of the further composition; and
administering material of the further composition to a person sufficiently to treat a medical condition of the person or/and to prevent the person from contracting the medical condition.

63. A composition comprising:
a liquid carrier;
active material dispersed largely throughout the carrier, the active material comprising salt of peroxymonosulfuric acid or/and reaction product of the salt of peroxymonosulfuric acid and other material of the composition, the salt of peroxymonosulfate having active oxygen consisting of chemically readily transferable oxygen atoms; and
an inhibitor dispersed largely throughout the carrier for inhibiting loss of active oxygen from the composition.

64. A composition as in claim 63 wherein the inhibitor causes the loss of active oxygen from the claimed composition to be at least 10% lower as a function of time than what would arise in a comparative composition lacking the inhibitor but otherwise constituted the same as the claimed composition up to a point at which the comparative composition has lost 50% of its active oxygen.

65. A composition as in claim 63 wherein the salt of peroxymonosulfuric acid comprises potassium hydrogen peroxymonosulfate.

66. A composition as in claim 63 wherein the active material further includes at least one of the following additional materials:
inorganic halide or/and reaction product of the inorganic halide and other material of the composition;
metal phosphate or/and reaction product of the metal phosphate and other material of the composition;
sulfamic acid or/and reaction product of sulfamic acid and other material of the composition;
a non-reducing organic acid or/and reaction product of the organic acid and other material of the composition; and
a surfactant or/and reaction product of the surfactant and other material of the composition.

67. A method comprising administering material of the composition of claim 63 to a person sufficiently to treat a medical condition of the person or/and to prevent the person from contracting the medical condition.

68. A composition comprising:
a liquid carrier comprising water;
active material dispersed largely throughout the carrier, the active material comprising (a) inorganic halide or/and reaction product of the inorganic halide and other material of the composition and (b) an oxidizing agent or/and reaction product of the oxidizing agent and other material of the composition, the oxidizing agent being reactable in water with the inorganic halide to generate hypohalite ions and having active oxygen consisting of chemically readily transferable oxygen atoms; and
an inhibitor dispersed largely throughout the carrier for inhibiting loss of active oxygen from the composition.

69. A composition as in claim 68 wherein the inhibitor causes the loss of active oxygen from the claimed composition to be at least 10% lower as a function of time than what would arise in a comparative composition lacking the inhibitor but otherwise constituted the same as the claimed composition up to a point at which the comparative composition has lost 50% of its active oxygen.

70. A composition as in claim 68 wherein the active material further includes at least one of the following additional materials:
metal phosphate or/and reaction product of the metal phosphate and other material of the composition;
sulfamic acid or/and reaction product of sulfamic acid and other material of the composition;
a non-reducing organic acid or/and reaction product of the organic acid and other material of the composition; and
a surfactant or/and reaction product of the surfactant and other material of the composition.

71. A method comprising administering material of the composition of claim 68 to a person sufficiently to treat a medical condition of the person or/and to prevent the person from contracting the medical condition.
